(19) United States
(12) Patent Application Publication
(10) Pub. No.: US 2025/0222066 A1
(43) Pub. Date: Jul. 10, 2025

(54) COMPOSITIONS AND METHODS OF TREATING, PREVENTING OR INHIBITING A FACULTATIVE ANAEROBE INFECTION

(71) Applicant: UNIVERSITY OF UTAH RESEARCH FOUNDATION, Salt Lake City, UT (US)

(72) Inventors: Nicholas N. ASHTON, Salt Lake City, UT (US); Dustin L. WILLIAMS, Salt Lake City, UT (US)

(21) Appl. No.: 18/879,504

(22) PCT Filed: Jun. 30, 2023
(86) PCT No.: PCT/US2023/026763

§ 371 Date: Dec. 27, 2024

Related U.S. Application Data
(60) Provisional application No. 63/357,041, filed on Jun. 30, 2022.

(51) Int. Cl. A61K 38/14; A61K 31/14; A61K 31/345; A61K 31/381; A61K 31/4168; A61K 31/4178; A61K 31/422; A61K 31/43; A61K 31/433; A61K 31/4439; A61K 31/454; A61K 31/496; A61K 31/517; A61K 31/53; A61K 31/5365; A61K 31/5377; A61K 31/5383; A61K 31/541; A61K 31/675; A61K 31/7036; A61P 31/04

(52) U.S. Cl. CPC A61K 38/14; A61K 31/14; A61K 31/345; A61K 31/381; A61K 31/4168; A61K 31/4178; A61K 31/422; A61K 31/43; A61K 31/433; A61K 31/4439; A61K 31/454; A61K 31/496; A61K 31/517; A61K 31/53; A61K 31/5365; A61K 31/5377; A61K 31/5383; A61K 31/541; A61K 31/675; A61K 31/7036; A61P 31/04

(57) ABSTRACT

Disclosed herein, are synergistic compositions comprising a nitroimidazole compound, a nitrofuran compound, or an anti-tumor agent; and a low minimum inhibitory concentration antibiotic, and methods of treating, preventing or inhibiting a facultative anaerobe infection by administering the compositions to a subject.

### CROSS-REFERENCE TO RELATED APPLICATIONS

This application claims the benefit of the filing date of U.S. Provisional Application No. 63/357,041 which was filed on Jun. 30, 2022. The content of this earlier filed application is hereby incorporated herein by reference in its entirety.

### STATEMENT REGARDING FEDERALLY FUNDED RESEARCH

This invention was made with government support under grant W81XWH-20-1-0069 awarded by The United States Army/MRMC. The government has certain rights in the invention.

### BACKGROUND

Biofilm-related infections are a major concern for military healthcare due to the traumatic nature of battlefield-relevant injuries in which bacterial contamination levels are high. Biofilms lead to biofilm-impaired wound healing in soft tissues and difficult to treat infections, for example, in open fractures. Antibiotic tolerance of biofilm bacterial exceeds the toxic upper thresholds of safe systemic antibiotic doses. Thus, new treatments are needed.

### SUMMARY

Disclosed herein are methods of treating, preventing or inhibiting a facultative anaerobe infection in a subject, the methods comprising administering to the subject one or more effective doses of a composition comprising a synergistic combination of a nitroimidazole compound and a low minimum inhibitory concentration (MIC) antibiotic.

Disclosed herein are methods of treating, preventing or inhibiting a facultative anaerobe infection in a subject, the methods comprising administering to the subject one or more effective doses of a composition comprising a synergistic combination of a nitrofuran compound; and a low minimum inhibitory concentration (MIC) antibiotic.

Disclosed herein are methods of treating, preventing or inhibiting a facultative anaerobe infection in a subject, the methods comprising administering to the subject one or more effective doses of a composition comprising a synergistic combination of an anti-tumor agent; and a low minimum inhibitory concentration (MIC) antibiotic.

Disclosed herein are methods of inhibiting, treating or preventing a facultative anaerobe infection in a subject, the methods comprising administering to the subject having said infection a plurality of therapeutically effective doses of a composition comprising a synergistic combination of a nitroimidazole compound; and a low minimum inhibitory concentration (MIC) antibiotic.

Disclosed herein are methods of inhibiting, treating or preventing a facultative anaerobe infection in a subject, the methods comprising administering to the subject having said infection a plurality of therapeutically effective doses of a composition comprising a synergistic combination of a nitrofuran compound; and a low minimum inhibitory concentration (MIC) antibiotic.

Disclosed herein are methods of inhibiting, treating or preventing a facultative anaerobe infection in a subject, the methods comprising administering to the subject having said infection a plurality of therapeutically effective doses of a composition comprising a synergistic combination of an anti-tumor agent; and a low minimum inhibitory concentration (MIC) antibiotic.

Disclosed herein are combination therapies comprising a nitrofuran compound; and a low minimum inhibitory concentration (MIC) antibiotic.

Disclosed herein are combination therapies comprising an anti-tumor agent; and a low minimum inhibitory concentration (MIC) antibiotic.

Disclosed herein are kits for use in treating a subject suffering from a facultative anaerobe infection, the kits comprising: (a) a nitroimidazole compound; and (b) a low minimum inhibitory concentration (MIC) antibiotic.

Disclosed herein are kits for use in treating a subject suffering from a facultative anaerobe infection, the kits comprising: (a) a nitrofuran compound; and (b) a low minimum inhibitory concentration (MIC) antibiotic.

Disclosed herein are kits for use in treating a subject suffering from a facultative anaerobe infection, the kits comprising: (a) an anti-tumor agent; and (b) a low minimum inhibitory concentration (MIC) antibiotic.

Disclosed herein are kits for use in inhibiting replication of a facultative anaerobe infection, the kits comprising: (a) a nitroimidazole compound; and (b) a low minimum inhibitory concentration (MIC) antibiotic.

Disclosed herein are kits for use in inhibiting replication of a facultative anaerobe infection, the kits comprising: (a) a nitrofuran compound; and (b) a low minimum inhibitory concentration (MIC) antibiotic.

Disclosed herein are kits for use in inhibiting replication of a facultative anaerobe infection, the kits comprising: (a) an anti-tumor agent; and (b) a low minimum inhibitory concentration (MIC) antibiotic.

Disclosed herein are compositions comprising: (a) a nitroimidazole compound, a nitrofuran compound, or an anti-tumor agent; and (b) a low minimum inhibitory concentration (MIC) antibiotic.

Disclosed herein are methods of reducing cumulative bioburden of a facultative anaerobe in a subject, the methods comprising administering to the subject one or more effective doses of a composition comprising a synergistic combination of a nitroimidazole compound; and a low minimum inhibitory concentration (MIC) antibiotic.

Disclosed herein are methods of reducing cumulative bioburden of a facultative anaerobe in a subject, the methods comprising administering to the subject one or more effective doses of a composition comprising a synergistic combination of a nitrofuran compound; and a low minimum inhibitory concentration (MIC) antibiotic.

Disclosed herein are methods of reducing cumulative bioburden of a facultative anaerobe in a subject, the methods comprising administering to the subject one or more effective doses of a composition comprising a synergistic combination of an anti-tumor agent; and a low minimum inhibitory concentration (MIC) antibiotic.

Disclosed herein are devices comprising: an implantable reservoir, the implantable reservoir having: a permeable membrane that defines an interior volume of the implantable reservoir; and an inlet port in fluid communication with the interior volume, wherein the inlet port is configured to permit delivery of an antibiotic composition into the interior volume of the implantable reservoir, and wherein the permeable membrane is configured to permit diffusion of the antibiotic composition from the interior volume of the implantable reservoir through the permeable membrane to an exterior of the implantable reservoir.

### BRIEF DESCRIPTION OF THE DRAWINGS

The accompanying figures, which are incorporated in and constitute a part of this specification, illustrate several aspects and together with the description serve to explain the principles of the invention.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

FIG. 1 shows biofilms grown using a CDC biofilm reactor on 0.5″ coupons with the indicated organism were treated with 2 mL solutions of CAMHB and the indicated antibiotics for 24 h. After treatment, the biofilm coupons were removed from the treatment broth, rinsed, and the bioburden quantified using an established method of vortexing, sonicating, and serial dilution plating (Blue bars). The bioburden in the broth treatments was also quantified by centrifuging the bacteria, rinsing, and suspending three times before 10-fold serial dilution plating (Red bars). The reactor controls (n=5) are represented by the black line and the S.D. by the gray shaded region. Error bars are S.D. where n=5.

FIG. 2 shows S. aureus ATCC 6538 biofilms grown using a CDC biofilm reactor on 0.5″ coupons with the indicated organism were treated with 2 ml solutions of CAMHB and 1 mM of the indicated antibiotic(s) for 24 h. After treatment, the cumulative bioburden on the coupons and in the treatment was quantified with an established method of centrifuging the bacteria, rinsing, and suspending three times before 10-fold serial dilution plating. The log₁₀ reduction was calculated from the reactor controls (n=4). The error bars give the S.D. for the propagated errors of the log reduction calculation (n=5). Experiments with nitroimidazoles are green, those with nitrofurans are blue, and those with anti-tumor agents are orange.

FIG. 3 shows P. aeruginosa ATCC 27853 biofilms grown using a CDC biofilm reactor on 0.5″ coupons with the indicated organism were treated with 2 ml solutions of CAMHB with the indicated antibiotic(s) for 24 h. Gentamicin was prepared at 0.1 mM and the other compounds at 1 mM. After treatment, the cumulative bioburden on the coupons and in the treatment, was quantified with an established method of centrifuging the bacteria, rinsing, and suspending three times before 10-fold serial dilution plating. The log₁₀ reduction was calculated from the reactor controls (n=4). The error bars give the S.D. for the propagated errors of the log reduction calculation (n=5). Experiments with nitroimidazoles are green, those with nitrofurans are blue, and those with anti-tumor agents are orange.

FIGS. 4A-B show the cumulative data for compound testing against S. aureus (FIG. 4A) and P. aeruginosa (FIG. 4B) from FIG. 2 and FIG. 3, respectively, are displayed together here for study compounds in β-lactam antibiotics nafcillin or cefepime (left) and gentamicin (right). For comparison, the purple line gives the average bioburden for the antibiotic controls (nafcillin, cefepime, or gentamicin) without the study compound. The shaded purple region represents±S.D.

FIGS. 5A-F show the histochemical staining of pimonidazole adducts in the biofilm core with the Hypoxyprobe™ kit. FIGS. 5A and 5C show the staining of pimonidazole adducts in pimonidazole-treated biofilms is localized in the core (arrow) in the deepest location adjacent to the collagen substrate (star). Regions where oxygen tension are insufficiently low for pimonidazole adduct formation is also shown (arrow head). FIGS. 5C and 5D show dense biofilm plumes developed off an acute edge of collagen substrate. FIGS. 5B and 5D) show that the samples which did not receive pimonidazole treatment, developed very little non-specific staining (brown), providing a good signal-noise ratio for the technique. FIGS. 5E and 5F show the structures of metronidazole and pimonidazole; both are hypoxia activated prodrugs of the nitroimidazole family of molecules.

FIG. 6 shows the potent radical-scavenger edaravone protects S. aureus biofilms from the killing power of metronidazole against biofilm phenotypes. S. aureus biofilms grown using a CDC biofilm reactor and treated with 2 ml solutions of CAMHB in 0.25 mg/ml nafcillin (serving as a bacteriostatic agent) and 4 mg/ml metronidazole (with herein established activity against biofilms) and the indicated concentration of the potent radical-scavenger edaravone (0-2 mg/ml). After treatment, the biofilm coupons were removed from the treatment broth, rinsed and the bioburden quantified using a method of vortexing, sonicating, and serial dilution plating. Serial concentrations of edaravone proportionally negate killing effects of metronidazole on S. aureus biofilms. This data shows that the mechanism of metronidazole involves a radical ion species, that if quenched, neutralizes its killing power (n=8 for each data bar).

FIG. 7 shows the colony forming units (CFU) remaining in S. aureus ATCC 6538 biofilms (n=5) after 24 h antibiotic treatment in vancomycin, nafcillin, gentamicin, vancomycin, metronidazole, or combinations thereof. Accompanying MIC values of each antibiotic or combination are provided. The MIC for metronidazole indicates it has little effect against planktonic bacteria.

FIG. 8 shows proposed mechanism of action of nitroimidazole compounds.

FIGS. 9A-B show biofilm histochemical staining. FIG. 9A shows regions with pimonidazole histochemical stain (light brown) and safranin counterstain (pink). Staining of pimonidazole adducts in pimonidazole-treated S. aureus biofilms is localized in the core; the deepest location (darker brown region indicated by arrow) adjacent to the collagen substrate. Regions where oxygen tension is insufficiently low for pimonidazole adduct formation is shown (arrow head; lighter brown region). FIG. 9B shows the live/dead imaging of a S. aureus biofilm treated for 24 h with 0.5 mg/ml metronidazole. Dead cells (red) resided deep within the biofilm core. Viable bacteria (green) resided on the biofilm surface, where oxygen and nutrients were readily available.

FIGS. 10A-B show that antibiotic powders sprinkled in wounds diminish quickly. FIG. 10A shows representative release curves of common local delivery products compared to sustained antibiotic delivery by the Pouch device disclosed herein. Pouch curves were obtained in-vitro using a flow chamber. Other release curves were adapted from literature references. FIG. 10B shows that from an in vivo sheep study using the Pouch device disclosed herein that percent of drug released each day as calculated from the device contents removed as part of daily refilling procedures (n=5).

FIGS. 11A-B show the Pouch device that can be used to deliver the compositions disclosed herein. FIG. 11A shows a schematic of the Pouch device represented in an appropriate application in a fracture fixation procedure. FIG. 11B shows a photograph of one embodiment of the Pouch device; showing a removable trocar facilitating device insertion.

FIGS. 12A-C show infection outcomes in a sheep model of biofilm-related musculoskeletal infection (n=5 sheep/group). FIG. 12A shows the bio-burden in local tissues for the indicated treatments. FIG. 12B shows the bioburden on site hardware for the indicated treatment. FIG. 12C shows the change in site bioburden from initial inocula for the indicated treatments. Data were gathered 21 days after the inoculation and surgical procedure. Bars represent the average; the error bars represent #S.D.

FIGS. 13A-F show representative biofilms grown on coupons in a CDC biofilm reactor. FIG. 13A shows a gross photograph of S. aureus biofilms stained with methylene blue. Plumes of biofilm are readily visible. FIG. 13B shows a scanning electron microscope (SEM) image of S. aureus biofilms on the same coupon as FIG. 13A. The yellow arrow indicates zoomed in region of FIG. 13C, which shows pluming, three-dimensional nature of S. aureus biofilms. FIG. 13D shows a gross photograph of P. aeruginosa biofilms stained with methylene blue. A smooth sheet of biofilm is visible. FIG. 13E shows a SEM image of P. aeruginosa biofilms on the same coupon as FIG. 13D. The yellow arrow indicates zoomed in region of FIG. 13F, which shows a more sheet-like structure of P. aeruginosa biofilms.

FIG. 14 shows the proximal medial aspect of a sheep tibia with two simulated fracture fixation plates secured to the sur-face of the bone. Biofilms are grown on each plate. The overlaid SEM image (black and white image in circle) is of S. aureus biofilm on a simulated fracture fixation plate.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile, cerebral spinal fluid) that contains cells or cell components. In some aspects, the sample can be taken from the brain, spinal cord, cerebral spinal fluid or blood.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for multiple sclerosis, such as, for example, prior to the administering step. In some aspects of the disclosed methods, the “patient” has been diagnosed with a need for treatment for inhibiting, treating or preventing a facultative anaerobe infection, such as, for example, prior to the administering step.

As used herein, the term “normal” refers to an individual, a sample or a subject that does not have a facultative anaerobe infection or does not have an increased susceptibility of developing inhibiting, treating or preventing a facultative anaerobe infection.

As used herein, the term “susceptibility” refers to the likelihood of a subject being clinically diagnosed with a disease. For example, a human subject with an increased susceptibility for a facultative anaerobe infection can refer to a human subject with an increased likelihood of a subject being clinically diagnosed with a facultative anaerobe infection.

As used herein, the term “comprising” can include the aspects “consisting of” and “consisting essentially of.”

As used herein, a “control” is a sample from either a normal subject or from tissue from a normal subject that does not have a facultative anaerobe infection.

As used herein, the terms “synergy”, “synergism” or “synergistic” mean more than the expected additive effect of a combination. A synergistic effect may be attained when the active ingredients are: (1) co-formulated and administered or delivered simultaneously in a combined, unit dosage formulation; (2) delivered by alternation or in parallel as separate formulations; or (3) by some other regimen.

As used herein, “treat” is meant to mean administer a compound or composition of the invention to a subject, such as a human or other mammal (for example, an animal model), that has a facultative anaerobe infection, in order to prevent or delay a worsening of the effects of the disease or condition, or to partially or fully reverse the effects of the disease.

As used herein, “prevent” is meant to mean minimize the chance that a subject who has an increased susceptibility for developing a facultative anaerobe infection or will develop a facultative anaerobe infection.

As used herein, the terms “inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels. Further, the terms, “inhibit” or “inhibiting” mean decreasing bacterial colonization from the amount of colonization that would occur without treatment and/or causing an infection to decrease. Inhibiting also include causing a complete regression of the colonization.

As used herein, “modulate” is meant to mean to alter, by increasing or decreasing.

As used herein, the term “facultative anaerobe” refers to a facultative anaerobic organism that makes ATP by aerobic respiration if oxygen is present, but is capable of switching to fermentation if oxygen is absent.

As used herein, “effective amount” of a compound is meant to mean a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, the phrase “minimum inhibitory concentration (MIC)” refers to the lowest concentration (e.g., in μg/mL) of an antibiotic that inhibits the growth of a given strain of bacteria. In some aspects, MIC can depend on the microorganism, the affected subject and the antibiotic itself. In some aspects, MIC can be determined by culturing microorganisms in liquid media or on plates of solid growth of the organism. For example, “low MIC” or “low MIC value” can indicate that less drug is needed for inhibiting growth of the organism. Thus, antibiotics with lower MIC values are more effective antimicrobial agents. For example, clinical antibiotics are screened using a MIC assay to determine a MIC value (FDA requirements for antibiotics, also the process perfumed in hospital laboratories for patient cultures) to determine if a compound is useful or not for a given infection. This standard (and the clinical reliance on the MIC assay) has made a low MIC value a de facto (or functional) definition of an antibiotic.

As used herein, “low minimum inhibitory concentration (MIC) antibiotic” or “low MIC antibiotic” are used interchangeably and refer to antibiotics that have a low MIC or clinically acceptable breakpoint concentration.

Military medical facilities currently support Wounded Warriors and veterans who suffer from a cohort of injuries that affect soft tissues and bones of the extremities (Owens, B. D., et al. J Ortho Trauma 21, 254-257 (2007)). Combat-related infections gradually decreased throughout the 19^{th} and 20^{th} centuries with the advent of aseptic techniques, surgical debridement, delayed would closure, skeletal traction, and antibiotic therapies. Despite these medical advances, infection in this antibiotic era is the greatest mortality risk for the Wounded Warrior who survives the first 3 hours from point of injury (Murray, C. K., et al. J Trauma 64, S221-231 (2008)). These infections are debilitating to survivors, frequently leading to loss of function and/or amputation as more than 70% of combat-related injuries are to the extremities (Owens, B. D., et al. J Ortho Trauma 21, 254-257 (2007)). Due to the traumatic nature of battlefield-relevant injuries where bacterial contamination levels can be high, biofilm-related infection is a major concern for military healthcare.

A biofilm strategy protects constituent bacteria from host immunity and clinical intervention in a variety of ways. These bacteria excrete sticky exopolysaccharides to form cohesive communal aggregates and adhesive attachments to surfaces such as devitalized tissues and orthopedic hardware (Costerton, J. W. Clin Orthop Relat Res 437, 7-11 (2005); and Costerton, J. W., et al. Sci Am 238, 86-95 (1978); this deranges phagocytic clearance by host leukocytes, which are unable to engulf even small bacterial aggregates if larger than their own diameter (10-12 μm) (Stewart, P. S. Pathog Dis 70, 212-218, (2014)). Established biofilm communities comprise diverse phenotypic populations, in part as a response to varied microenvironments in the community. As the biofilm grows and matures, nutrients and oxygen become diffusion limited within the core, which by necessity becomes hypoxic and more metabolically quiescent than the biofilm surface which more aggressively consumes available oxygen and nutrients (Wessel, A. K. et al. mBio 5, e00992, (2014)). Conspicuously, the most prolific opportunistic pathogens in orthopedic infections are biofilm-forming facultative anaerobes; these are capable of both aerobic and anaerobic metabolism and include but not limited to Staphylococcus aureus, Staphylococcus epidermidis, Pseudomonas aeruginosa, Klebsiella pneumonia, and Acinetobacter baumannii among many others. Quiescent biofilm phenotypes can survive antibiotic concentrations much higher than tolerated by actively-replicating (planktonic) bacteria of the same genotype (Borriello, G. et al. Antimicrob Agents Chemother 48, 2659-2664 (2004); and Walters, M. C., 3rd, et al. Antimicrob Agents Chemother 47, 317-323, (2003)). The term “tolerance” is used herein to describe these phenotypic changes in contrast with the term “resistance” which describes the genotypic changes fueling the antibiotic-resistance epidemic. Because clinical antibiotics target metabolic pathways, they are less effective against phenotypes in the biofilm core which have diminished metabolic activity. These antibiotic tolerant phenotypes constitute ˜1-20% of biofilm bacteria with higher ratios in bulky more mature biofilms (Stewart, P. S. & Costerton, J. W. Lancet 358, 135-138 (2001)). They are a nidus of infection accounting for the notable recalcitrance of biofilm infections. Some authors refer to these tolerant phenotypes as ‘persister cells’ because they persist through the most rigorous systemic courses of clinical antibiotic chemotherapy to subsequently reseed infection (Lewis, K. Annu Rev Microbiol 64, 357-372 (2010); and Lewis, K. Handb Exp Pharmacol, 121-133 (2012)). There are limited treatment options to deal with biofilms that underpin these clinical infections.

Described herein are compositions and methods for targeting antibiotic-tolerant phenotypes in the recalcitrant biofilm core—an advancement in treating, preventing or inhibiting musculoskeletal infections.

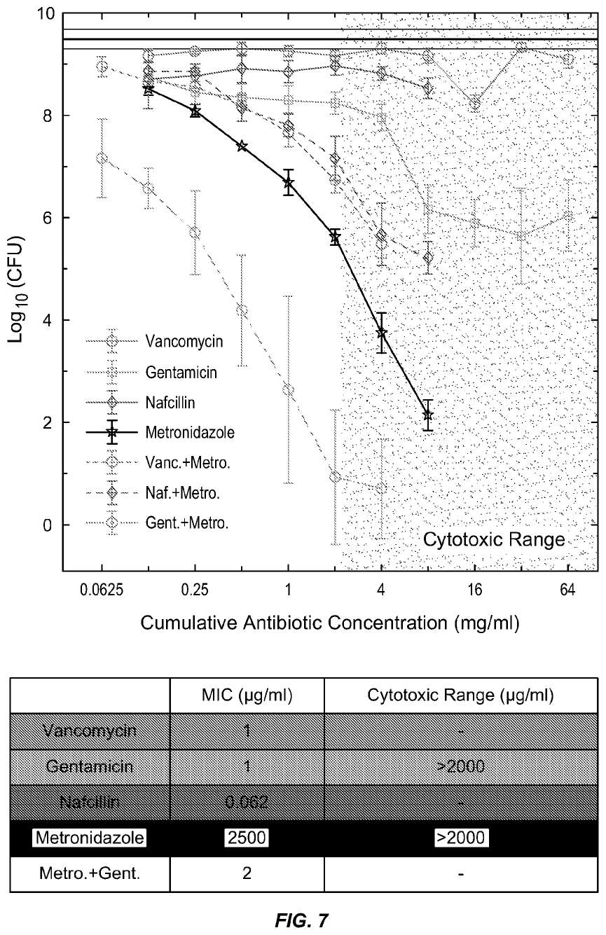

As described herein, testing with S. aureus biofilms, it was observed that the hypoxia-activated prodrug, metronidazole, displayed much greater antibiofilm activity compared to traditional antibiotics, an effect which considerably strengthened when paired adjunctively with gentamicin or levofloxacin but not with the β-lactam nafcillin (FIG. 7). Interestingly, ascending concentrations of clinical antibiotics—that represent the current standard of care—tested against biofilms displayed diminishing-returns in efficacy; an effect which plateaued even at inordinate concentrations (FIG. 7). In contrast, formulations with metronidazole displayed a graded concentration-dependent effect against biofilms (FIG. 7). Taken together, these data show that there are diminishing returns for antibiofilm strategies that attempt to deliver local, high doses of traditional antibiotics clinically that are now the standard of care: powdered antibiotics applied directly to the wound (vancomycin and tobramycin), antibiotic loaded bone cements and bone-void fillers, coatings, local infusion, etc. Disclosed herein is the use of, for example, metronidazole, as an adjunctive agent in local high-dose therapy to leverage its dose-dependent antibiofilm effect.

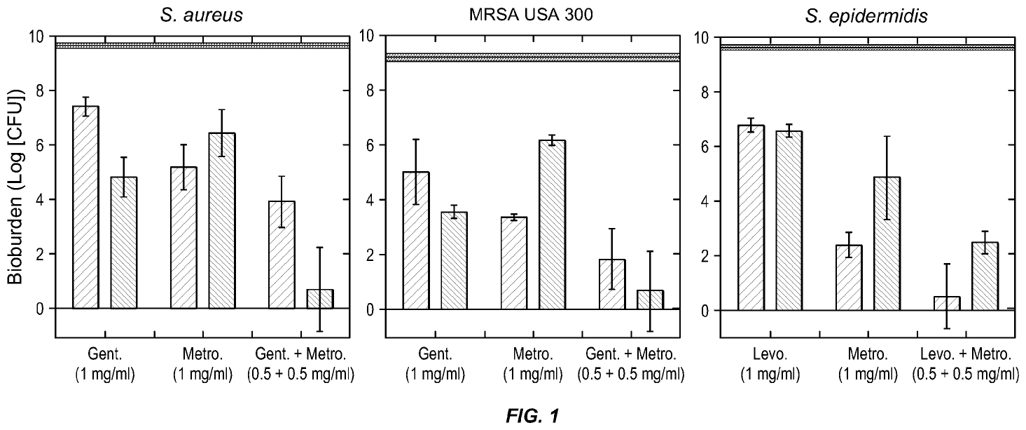

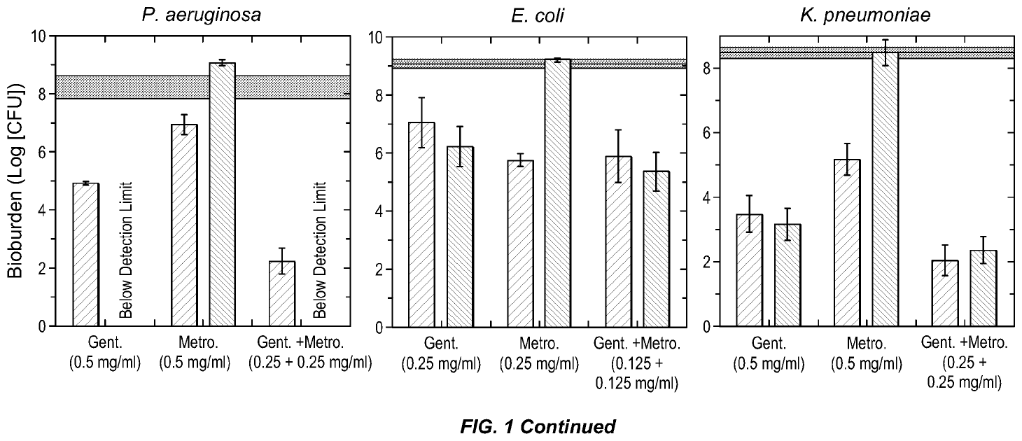

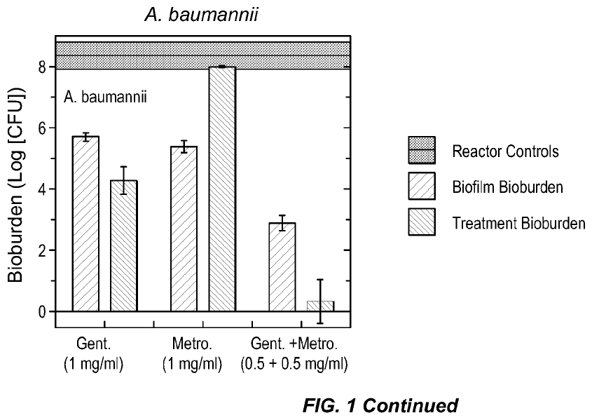

Managing biofilm-related infections poses a considerable challenge to clinicians despite the abundant availability of therapeutic compounds in this antibiotic era. Biofilms underpin these most difficult-to-treat infections types including, for example, implant-related infections, osteomyelitis, and chronic skin ulcers. Infectious bacteria undergo phenotypic changes as they form matrix-enclosed biofilm communities and become tolerant of traditional antibiotics. It is well established that mature biofilms tolerate antibiotic concentrations several orders of magnitude greater than indicated by the minimum inhibitory concentration (MIC) assay, which has long served as the gold-standards for determining clinical antimicrobial efficacy (FIG. 1). For example, vancomycin and gentamicin failed to eradicate S. aureus biofilms even at inordinate concentrations over 60,000 X the MIC, 6,000 X the nephrotoxic and ototoxic thresholds, and 25-50 X the cytotoxic limit (FIG. 1). Because the MIC assay is performed on planktonic phenotypes, it does not accurately represent the ability of an antibiotic to kill the biofilms underpinning these difficult-to-treat infections.

As described herein, nitroimidazole compounds were identified as an antibiofilm agent when used in combination with traditional, low-MIC antibiotics (FIG. 1). Clinically, nitroimidazole compounds are indicated against anaerobic bacteria and do not have acceptable MICs against facultative anaerobes, such as S. aureus (2500 μg/ml). Facultative anaerobes include most of the prolific biofilm forming pathogens (e.g., E. coli, S. aureus, P. aeruginosa, S. epidermidis, and Streptococcus sp). Facultative anaerobes can shift phenotypic behavior to fit anaerobic environments as might be the case inside biofilms and wounds which are both known to have high internal oxygen tension. The MIC testing used clinically to determine antimicrobial efficacy ignores these phenotypic distinctions while simultaneously ignoring the biofilm phenotypes which underpin these difficult-to-treat infections. Treating the biofilm with metronidazole (a nitroimidazole compound) killed the otherwise difficult-to-kill persister cells embedded within the biofilm (FIG. 1). Targeting both anaerobic and aerobic phenotypes simultaneously in S. aureus biofilms showed strong synergy when combining gentamicin with metronidazole (FIG. 1). Screening antibiotics successfully identified Nitroimidazoles combinations with much greater efficacy against S. aureus biofilms than two gold-standard antibiotics alone (vancomycin and gentamicin). Nitroimidazole combinations demonstrate near-complete biofilm eradication below cytotoxic concentrations (FIG. 1, Cyan trace) providing a scientific basis for using these combinations in clinical products and therapies against the difficult-to-treat biofilm-related infections. Products that use these combinations include topical gels and ointments, eye drops, ear drops, nasal sprays, and systemically administered IV or oral systems.

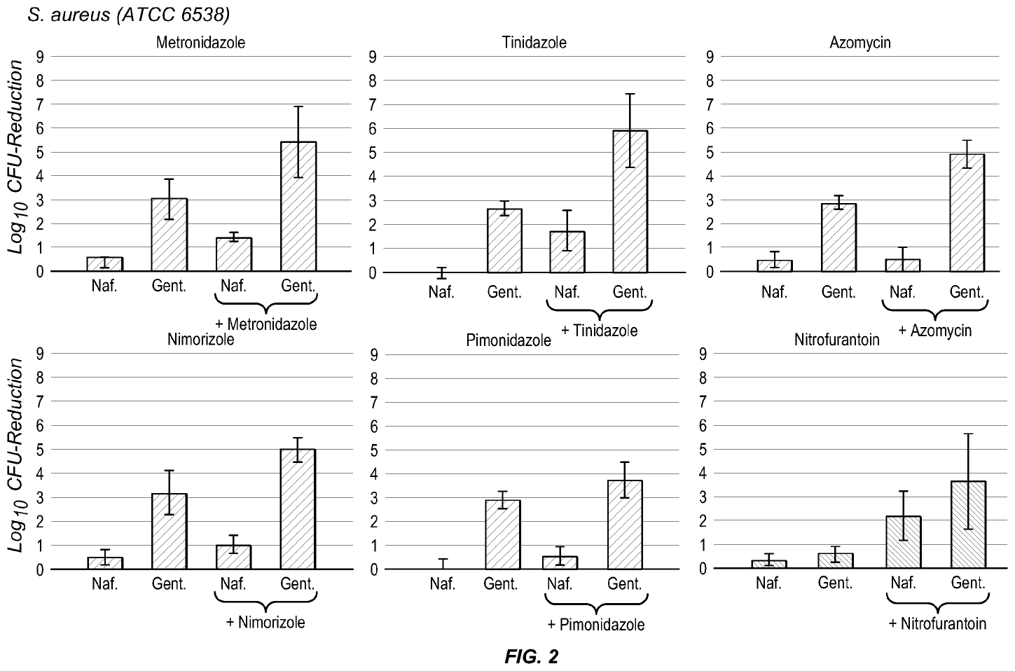

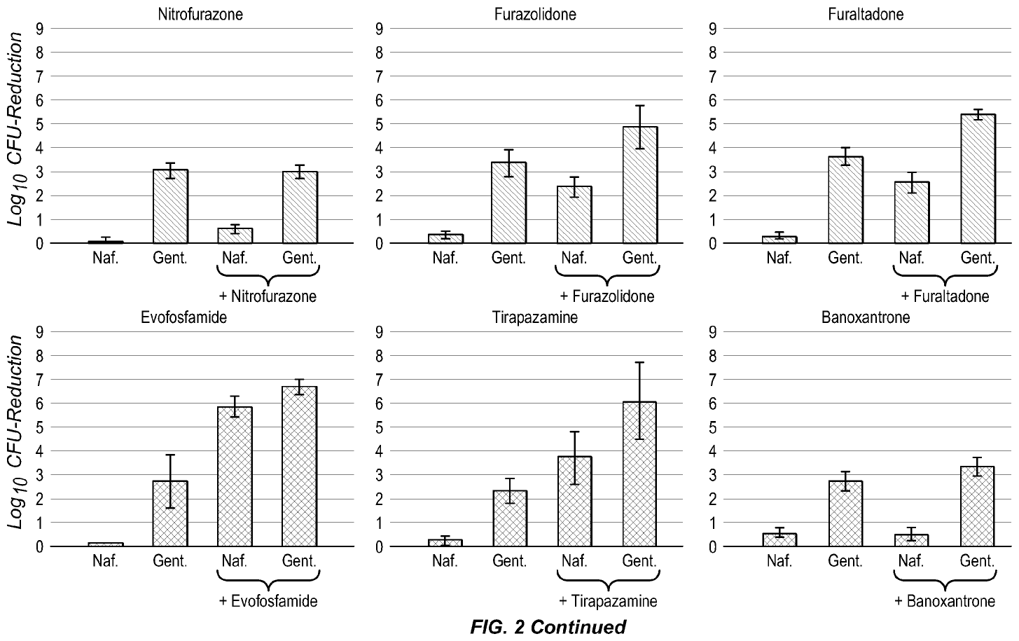

For example, a topical formulation was tested using an in-vitro model with biofilms grown on college. A metronidazole and gentamycin (0.1 and 0.1%) combination matched or exceeded the clinically relevant topicals tested (FIG. 2). In sum, these results demonstrate a superior composition with activity against the biofilms underpinning the worst clinical infections.

The compositions and methods disclosed herein can be used in products such as topical antibiotic gels or ointments, eye drops, ear drops, nasal sprays, and systemically administered IV or oral therapeutics. These antimicrobial combinations will also be useful in the development of antimicrobial coatings, impregnated plastics, and implantable antibiotic hydrogels.

### Compositions

Disclosed herein are compositions comprising: (a) a nitroimidazole compound, a nitrofuran compound, or an anti-tumor agent; and (b) a low minimum inhibitory concentration (MIC) antibiotic. In some aspects, the nitroimidazole compound can be metronidazole, tinidazole, nimorizole, pimonidazole, or nimorizole. In some aspects, the nitrofuran compound can be furaltadone, nitrofurantoin, nitrofurazone, or furazolidone. In some aspects, the anti-tumor agent can evofosfamide and tirapazamine, or banoxanthrone. In some aspects, the low MIC antibiotic can be gentamicin, nafcillin, levofloxacin, or vancomycin.

In some aspects, the composition can be formulated as or formulated for: a capsule, a tablet, a gel, a geltab, a liquid, a solid, an elixir, a spray, a powder, a suppository or an implant, a sachet, a lozenge, a freeze-dried composition, or a hydrogel. In some aspects, the composition can be impregnated into a plastic material.

Also disclosed herein are compositions comprising a nitroimidazole compound, a nitrofuran compound, and/or an anti-tumor agent; and a low minimum inhibitory concentration (MIC) antibiotic. As further described herein, a nitroimidazole compound, a nitrofuran compound, or an anti-tumor agent; and a low minimum inhibitory concentration (MIC) antibiotic can act synergistically to reduce the cumulative bacterial bioburden of the biofilm phenotypes. Synergy between antibiotics, can be determined by any suitable method using bacterial biofilms or non log-phase replicating phenotypes. Methods includes minimum bactericidal concentration (MBC) testing, biofilm testing using various reactor systems. The CDC-biofilm reactor, the calgary biofilm device are the most common for this testing.

Disclosed herein are combination therapies comprising a nitroimidazole compound; and a low minimum inhibitory concentration (MIC) antibiotic. In some aspects, the nitroimidazole compound and the low MIC antibiotic can be provided in a single formulation. In some aspects, the nitroimidazole compound and the low MIC antibiotic can be provided separately.

Disclosed herein are combination therapies comprising a nitrofuran compound; and a low minimum inhibitory concentration (MIC) antibiotic. In some aspects, the nitrofuran compound and the low MIC antibiotic can be provided in a single formulation. In some aspects, the nitrofuran compound and the low MIC antibiotic are provided separately.

Disclosed herein are combination therapies comprising an anti-tumor agent; and a low minimum inhibitory concentration (MIC) antibiotic. In some aspects, the anti-tumor agent and the low MIC antibiotic can be provided in a single formulation. In some aspects, the anti-tumor agent and the low MIC antibiotic can be provided separately.

Nitroimidazole compounds, nitrofuran compounds and anti-tumor agents described herein can act as hypoxia activated prodrugs that increase the bactericidal efficacy of antibiotics, for example, traditional antibiotics (e.g., antibiotics with low MIC values). In some aspects, a nitroimidazole compound, a nitrofuran compound or an anti-tumor agent can increase the killing capacity when combined with a low MIC antibiotic

Each of the nitroimidazole compounds, nitrofuran compounds, anti-tumor agents, and low MIC antibiotics can be administered by any suitable route of administration. For example, and without limitation, each antibiotic may independently be administered intravenously, orally, parenterally, subcutaneously, by inhalation, by injection, and/or by infusion.

Each of the nitroimidazole compounds, nitrofuran compounds, anti-tumor agents, and low MIC antibiotics in the combination therapy can be administered simultaneously or sequentially. Sequential administration or alternating administration can include providing each of the components exclusively for a period of time. Sequential administration can include a period of overlap in which the subject is provided both the IV formulation containing nitroimidazole compounds, nitrofuran compounds, anti-tumor agents and the formulation containing the low MIC antibiotic. The periods of exclusivity and periods of overlap can independently be 1 hour, 2 hours, 3 hours, 4 hours, 5 hours, 6 hours, 1 day, 2 days, 3 days, 4 days, 5 days, 6 days, 1 week or 2 weeks.

One or more of the components in the combination therapies can be administered to the subject using the same dosing regimen. One or more of the components in the combination therapies can be administered according to different dosing regimens. A dosing regimen can include a dosage, a schedule or administration, or both. A dosage can be described by an absolute amount of drug (e.g., mg), or by a relative amount of the drug to the subject (e.g., mg/kg). A schedule of administration can be described by the interval between doses. For example and without limitation, the interval between doses can be about an hour, about 2 hours, about 3 hours, about 4 hours, about 6 hours, about 8 hours, about 12 hours, about 24 hours, about 36 hours, about 48 hours, about 3 days, about 4 days, about 5 days, about 6 days, about 7 days, or more.

One or more of the components in the combination therapies can be provided in a single formulation. One or more of the components in the combination therapies can be provided in separate formulations. Each formulation can be prepared for delivery by a particular route of administration, such as intravenously, orally, parenterally, subcutaneously, by inhalation, by injection, and/or by infusion.

Any of the nitroimidazole compounds, nitrofuran compounds, anti-tumor agents, and low MIC antibiotics can be provided as pharmaceutically acceptable salts, such as nontoxic acid addition salts, which are salts of an amino group formed with inorganic acids such as but not limited to hydrochloric acid, hydrobromic acid, phosphoric acid, sulfuric acid and perchloric acid or with organic acids such as, but not limited to, acetic acid, maleic acid, tartaric acid, citric acid, succinic acid, methansulfonic acid, glucuronic acid, malic acid, gluconic acid, lactic acid, aspartic acid, or malonic acid.

The formulation can be administered by injection, infusion, implantation (intravenous, intramuscular, subcutaneous, or the like) or by inhalation in dosage forms, formulations, or via suitable delivery devices or implants containing conventional, non-toxic pharmaceutically acceptable carriers, solvents, diluents, and adjuvants. The formulation and preparation of such compositions are well known to those skilled in the art of pharmaceutical formulation.

Formulations for parenteral use can be provided in unit dosage forms (e.g., in single-dose ampoules and vials), in vials containing several doses and in which a suitable preservative can be added (see below), in prefilled syringes, or in prefilled IV bags. The pharmaceutical compositions described herein can be in the form suitable for sterile injection. Formulations can be formulated for parenteral administration, such as by injection or infusion. In some aspects, the injection or infusion can be subcutaneous or intravenous.

Depending upon the needs of the patient, and the clinical conditions, administration of the composition by IV administration can be favored over oral administration because it allows for rapid introduction of the antibiotic into systemic circulation, provides complete bioavailability, allows to better control the pharmacokinetic parameters that are driving the pharmacological efficacy, and avoids issues of stability in the gastrointestinal tract and absorption.

### Pharmaceutical Compositions

As disclosed herein, are pharmaceutical compositions, comprising one or more of the compositions, synergistic compositions or combination therapies disclosed herein. As disclosed herein, are pharmaceutical compositions, comprising a nitroimidazole compound and a low MIC antibiotic; a nitrofuran compound and a low MIC antibiotic; or an anti-tumor agent and a low MIC antibiotic. In some aspects, any of the pharmaceutical compositions can also comprise a pharmaceutical acceptable carrier. In some aspects, imipramine can be formulated for oral or parental administration. In some aspects, the parental administration is intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

In some aspects, the pharmaceutical compositions can further comprise one or more pharmaceutically acceptable excipients, diluents, adjuvants, stabilizers, emulsifiers, preservatives, colorants, buffers, flavor-imparting agents. In some aspects, the pharmaceutical compositions can further comprise one or more pharmaceutically acceptable carriers suitable for oral, enteral, mucosal, sub-mucosal, parenteral, intravenous, nasal, ocular, ear or transdermal administration.

In some aspects, the compositions can be formulated for intravenous injection, intraperitoneal injection or implantation, intramuscular injection or implantation, intrathecal injection, subcutaneous injection or implantation, intradermal injection, lavage, bladder wash-out, suppositories, pessaries, oral ingestion, topical application, enteric application, inhalation, aerosolization or nasal spray or drops, ocular administration, or administration to the ear.

In some aspects, the compositions or pharmaceutical compositions can be manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation. In some aspects, the compositions or pharmaceutical compositions can be formulated as an implantable hydrogel or a hydrogel for transdermal application.

The compositions and pharmaceutical compositions disclosed herein can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions and pharmaceutical compositions disclosed herein can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes c dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, a pharmaceutical composition comprises a nitroimidazole compound; and a low MIC antibiotic; a nitrofuran compound; and a low MIC antibiotic; or an anti-tumor agent; and a low MIC antibiotic; and optionally, a pharmaceutical acceptable carrier. Further, the pharmaceutical composition comprises a nitroimidazole compound; and a low MIC antibiotic; a nitrofuran compound; and a low MIC antibiotic; or an anti-tumor agent; and a low MIC antibiotic in therapeutically effective amounts. In some aspects, the nitroimidazole compound can be metronidazole, tinidazole, nimorizole, pimonidazole, or nimorizole. In some aspects, the nitrofuran compound can be furaltadone, nitrofurantoin, nitrofurazone, or furazolidone. In some aspects, the anti-tumor agent can evofosfamide and tirapazamine, or banoxanthrone. In some aspects, the low MIC antibiotic can be gentamicin, nafcillin, levofloxacin, or vancomycin. In some aspects, the pharmaceutical composition can be formulated for oral or intravenous administration.

### Methods

Disclosed herein are methods of treating, preventing or inhibiting a facultative anaerobe infection in a subject. In some aspects, the methods can comprise administering to the subject one or more effective doses of a composition comprising a synergistic combination of a nitroimidazole compound and a low minimum inhibitory concentration (MIC) antibiotic.

Disclosed herein are methods of treating, preventing or inhibiting a facultative anaerobe infection in a subject. In some aspects, the methods can comprise administering to the subject one or more effective doses of a composition comprising a synergistic combination of a nitrofuran compound and a low minimum inhibitory concentration (MIC) antibiotic.

Disclosed herein are methods of treating, preventing or inhibiting a facultative anaerobe infection in a subject. In some aspects, the methods comprising administering to the subject one or more effective doses of a composition comprising a synergistic combination of an anti-tumor agent and a low minimum inhibitory concentration (MIC) antibiotic.

Disclosed herein are methods of inhibiting, treating or preventing a facultative anaerobe infection in a subject. In some aspects, the methods can comprise administering to the subject having said infection a plurality of therapeutically effective doses of a composition comprising a synergistic combination of a nitroimidazole compound and a low minimum inhibitory concentration (MIC) antibiotic. In some aspects, the plurality of therapeutically effective doses of the composition comprising a synergistic combination of the nitroimidazole compound and the low MIC antibiotic can be one or more doses administered per day for two or more days per week. In some aspects, dosing can be continued for one or more weeks per month. In some aspects, dosing can be continued for one or more months per year. In some aspects, the composition comprising a synergistic combination of the nitroimidazole compound and the low MIC antibiotic can be administered to the subject immediately after infection or any time within one day to 5 days after infection or at the earliest time after diagnosis of infection with a facultative anaerobe.

Disclosed herein are methods of inhibiting, treating or preventing a facultative anaerobe infection in a subject. In some aspects, the methods can comprise administering to the subject having said infection a plurality of therapeutically effective doses of a composition comprising a synergistic combination of a nitrofuran compound and a low minimum inhibitory concentration (MIC) antibiotic. In some aspects, the plurality of therapeutically effective doses of the composition comprising a synergistic combination of the nitrofuran compound and the low MIC antibiotic can be one or more doses administered per day for two or more days per week. In some aspects, dosing can be continued for one or more weeks per month. In some aspects, dosing can be continued for one or more months per year.

In some aspects, the composition comprising a synergistic combination of the nitrofuran compound and the low MIC antibiotic can be administered to the subject immediately after infection or any time within one day to 5 days after infection or at the earliest time after diagnosis of infection with a facultative anaerobe.

Disclosed herein are methods of inhibiting, treating or preventing a facultative anaerobe infection in a subject. In some aspects, the methods can comprise administering to the subject having said infection a plurality of therapeutically effective doses of a composition comprising a synergistic combination of an anti-tumor agent; and a low minimum inhibitory concentration (MIC) antibiotic. In some aspects, the plurality of therapeutically effective doses of the composition comprising a synergistic combination of the anti-tumor agent and the low MIC antibiotic can be one or more doses administered per day for two or more days per week. In some aspects, dosing can be continued for one or more weeks per month. In some aspects, dosing can be continued for one or more months per year. In some aspects, the composition comprising a synergistic combination of the anti-tumor agent and the low MIC antibiotic can be administered to the subject immediately after infection or any time within one day to 5 days after infection or at the earliest time after diagnosis of infection with a facultative anaerobe.

Disclosed herein are methods of reducing cumulative bioburden of a facultative anaerobe in a subject. In some aspects, the methods comprising administering to the subject one or more effective doses of a composition comprising a synergistic combination of a nitroimidazole compound and a low minimum inhibitory concentration (MIC) antibiotic.

Disclosed herein are methods of reducing cumulative bioburden of a facultative anaerobe in a subject. In some aspects, the methods comprising administering to the subject one or more effective doses of a composition comprising a synergistic combination of a nitrofuran compound and a low minimum inhibitory concentration (MIC) antibiotic.

Disclosed herein are methods of reducing cumulative bioburden of a facultative anaerobe in a subject. In some aspects, the methods comprising administering to the subject one or more effective doses of a composition comprising a synergistic combination of an anti-tumor agent and a low minimum inhibitory concentration (MIC) antibiotic.

In some aspects, the nitroimidazole compound can be metronidazole, azomycin, secnidazole, ornidazole, dimetriazole, panidazole, fexinidazole, benznidaole, misonidazole, magazol, tinidazole, nimorizole, pretomanid, delamanid, or pimonidazole.

In some aspects, the nitrofuran compound can be difurazone, furazolidone, nifurfoline, nifuroxazide, nifurquinazol, nifurtoinol, nifurzide, nitrofural, furaltadone, ranbezolid, furazidine, furaginum, furylfuramide, nifuratel, nifurtimox, nitrofurantoin, nitrofurazone, or furazolidone.

In some aspects, the anti-tumor agent can evofosfamide, tirapazamine, or banoxanthrone.

In some aspects, the low MIC antibiotic can be gentamicin, nafcillin, levofloxacin, or vancomycin.

In some aspects, the facultative anaerobe infection can be due to a microorganism. In some aspects, the microorganism can be a bacterium, a fungus or a parasite. In some aspects, the bacterium can be an anaerobe. In some aspects, the anaerobe can be Staphylococcus spp, or Pseudomonas spp.

In some aspects, the bacterium can be a Gram-negative bacterium. In some aspects, the Gram-negative bacterium can be E. coli, K. pneumoniae, A. baumannii, Proteus, S. typhimurium, B. subtilis; S. pyogenes; S. pneumoniae; E. faecalis; methicillin-resistant S. aureus; S. epidermidis or P. aeruginosa. In some aspects, the Gram-negative bacterium is not E. coli.

In some aspects, the Gram-negative bacterium is not E. coli and the nitroimidazole compound is not metronidazole. In some aspects, the facultative anaerobe infection is not in the bladder.

In some aspects, the bacterium can be a gram-positive bacterium. In some aspects, the gram-positive bacterium can be S. aureus or S. epidermidis.

Disclosed herein are methods of treating, preventing or inhibiting a S. Aureus infection in a subject, the methods comprising administering to the subject one or more effective doses of a composition comprising a synergistic combination of metronidazole and gentamicin or nafcillin.

Disclosed herein are methods of treating, preventing or inhibiting a S. Aureus infection in a subject, the methods comprising administering to the subject one or more effective doses of a composition comprising a synergistic combination of tinidazole and gentamicin or nafcillin.

Disclosed herein are methods of treating, preventing or inhibiting a S. Aureus infection in a subject, the methods comprising administering to the subject one or more effective doses of a composition comprising a synergistic combination of azomycin and gentamicin or nafcillin.

Disclosed herein are methods of treating, preventing or inhibiting a S. Aureus infection in a subject, the methods comprising administering to the subject one or more effective doses of a composition comprising a synergistic combination of nimorizole and gentamicin or nafcillin.

Disclosed herein are methods of treating, preventing or inhibiting a S. Aureus infection in a subject, the methods comprising administering to the subject one or more effective doses of a composition comprising a synergistic combination of pimonidazole and gentamicin or nafcillin.

Disclosed herein are methods of treating, preventing or inhibiting a S. Aureus infection in a subject, the methods comprising administering to the subject one or more effective doses of a composition comprising a synergistic combination of nitrofurantoin and gentamicin or nafcillin.

Disclosed herein are methods of treating, preventing or inhibiting a S. Aureus infection in a subject, the methods comprising administering to the subject one or more effective doses of a composition comprising a synergistic combination of nitrofurazone and gentamicin or nafcillin.

Disclosed herein are methods of treating, preventing or inhibiting a S. Aureus infection in a subject, the methods comprising administering to the subject one or more effective doses of a composition comprising a synergistic combination of furazolidone and gentamicin or nafcillin.

Disclosed herein are methods of treating, preventing or inhibiting a S. Aureus infection in a subject, the methods comprising administering to the subject one or more effective doses of a composition comprising a synergistic combination of furaltadone and gentamicin or nafcillin.

Disclosed herein are methods of treating, preventing or inhibiting a S. Aureus infection in a subject, the methods comprising administering to the subject one or more effective doses of a composition comprising a synergistic combination of evofosfamide and gentamicin or nafcillin.

Disclosed herein are methods of treating, preventing or inhibiting a S. Aureus infection in a subject, the methods comprising administering to the subject one or more effective doses of a composition comprising a synergistic combination of tirapazamine and gentamicin or nafcillin.

Disclosed herein are methods of treating, preventing or inhibiting a S. Aureus infection in a subject, the methods comprising administering to the subject one or more effective doses of a composition comprising a synergistic combination of banoxantrone and gentamicin or nafcillin.

Also disclosed herein are methods of treating a surface or product to kill or inhibit a facultative anaerobe. In some aspects, the method can comprise applying a composition comprising: (a) a nitroimidazole compound, a nitrofuran compound, or an anti-tumor agent; and (b) a low MIC antibiotic to a surface or product preserves the surface or product by preventing or inhibiting facultative anaerobe formation or growth by one or more facultative anaerobes. In some aspects, composition can be formulated as or formulated for: a capsule, a tablet, a gel, a geltab, a liquid, a solid, an elixir, a spray, a powder, a suppository or an implant, a sachet, a lozenge, a freeze-dried composition, or a hydrogel.

In some aspects, the surface can be selected from the group consisting of: microcapsules, catheters, wound dressings, implants, wound closures, staples, meshes, controlled drug delivery systems, wound coverings, fillers, sutures, tissue adhesives, tissue sealants, absorbable and non-absorbable hemostats, catheters, wound drainage tubes, arterial grafts, soft tissue patches, gloves, shunts, stents, guide wires and prosthetic devices, contact lens and contact lens storage containers, medical devices, oral swabs, sponges, skin swabs, dental appliances and dental devices, microbial-resistant fabrics and apparel, anti-microbial condoms, surgical gowns, microbial-resistant hospital equipment, anti-microbial paper products, animal care products, antimicrobial plastics, antimicrobial plastic devices, rubbers, appliances with antimicrobial constituents or coatings, food processing equipment, food conveyor belts, food packaging equipment, pet or animal food, pet chew toys, pet or animal water bowls, and floating toys.

In some aspects, the product can be selected from the group consisting of: lipsticks, cosmetics and personal care items, fabric detergents, dish detergents, cleansers, soaps, bubble baths, disinfectants, deodorizers, human and animal foods, beverages, antimicrobial packaging, pharmaceutical products feminine hygiene compositions, vaginal douches, antimicrobial soaps, hand sanitizers, deodorants, antiperspirants dental compositions, toothpastes, mouth rinses and washes medications, athlete's foot treatments, cold sore treatments, herpes virus treatments, medicated chewing gums, wound care compositions, dermatological compositions, acne treatments, skin conditioners, skin moisturizers, anti-wrinkle formulations, skin whiteners, sunscreens, tanning lotions, hair products, shampoos, shower gels, bubble baths, conditioners, shaving creams, spermicides.

In some aspects, in any of the methods disclosed herein, the subject has been identified as being in need of the treatment. In some aspects, the subject is infected or has previously been infected with a Gram-negative bacterium or a Gram-positive bacterium.

In some aspects, the subject can be a human.

In some aspects, the step of administering any of the compositions disclosed herein can comprise intravenous injection, intraperitoneal injection or implantation, intramuscular injection or implantation, intrathecal injection, subcutaneous injection or implantation, intradermal injection, lavage, bladder wash-out, suppositories, pessaries, oral ingestion, topical application, enteric application, inhalation, aerosolization or nasal spray or drops, ocular administration, or administration to the ear of the subject.

In some aspects, therapeutically effective dose of the nitroimidazole compound and low MIC antibiotic can be in a 1:1 ratio. In some aspects, the therapeutically effective dose of the nitrofuran compound and low MIC antibiotic is in a 1:1 ratio. In some aspects, the therapeutically effective dose of the anti-tumor agent and low MIC antibiotic can be in a 1:1 ratio.

### Devices

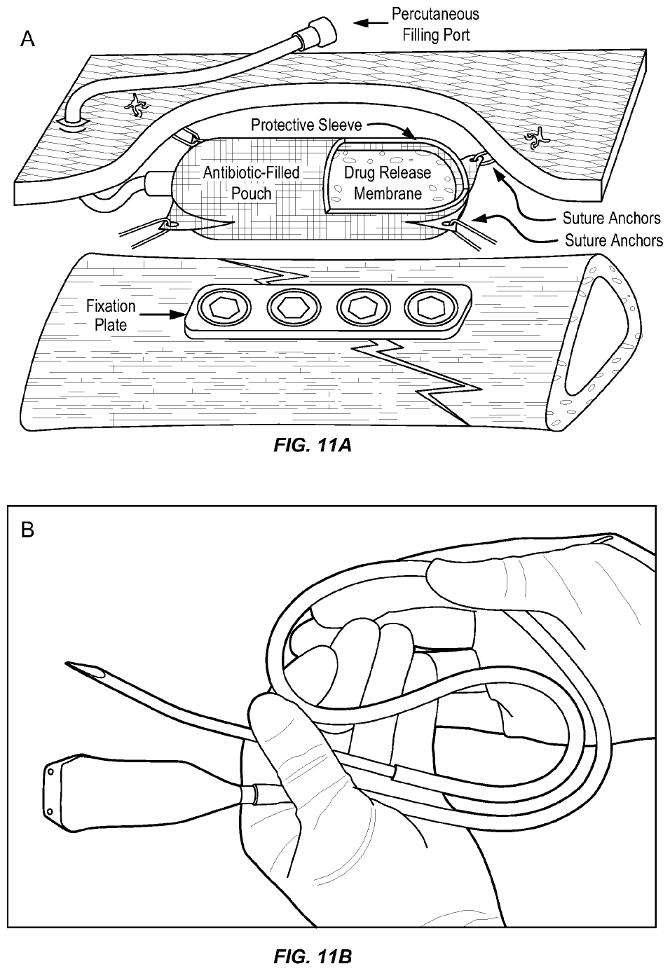

Disclosed herein are devices capable of administering to the subject one or more effective doses of a composition comprising a synergistic combination of a nitroimidazole compound and a low MIC antibiotic. In some aspects, the device can comprise an implantable reservoir, the implantable reservoir having: a permeable membrane that defines an interior volume of the implantable reservoir; and an inlet port in fluid communication with the interior volume, wherein the inlet port is configured to permit delivery of an antibiotic composition into the interior volume of the implantable reservoir, and wherein the permeable membrane is configured to permit diffusion of the antibiotic composition from the interior volume of the implantable reservoir through the permeable membrane to an exterior of the implantable reservoir. In some aspects, the device can further comprise at least one suture coupled to the implantable reservoir and configured to secure the implantable reservoir to a body of a subject. In some aspects, the device can further comprise a conduit having a first end in fluid communication with the inlet port. In some aspects, the device can further comprise a filling port, wherein the filling port is in fluid communication with a second end of the conduit. In some aspects, the filling port can be a percutaneous filling port. In some aspects, the devices described herein are referred to as a “Pouch device”. In some aspects, the pouch device disclosed herein can be the device illustrated in FIG. 11A or FIG. 11B. In some apsects, the disclosed devices can be pre-loaded with a nitroimidazole compound and a low minimum inhibitory concentration (MIC) antibiotic. In some apsects, the disclosed devices can be pre-loaded with nitrofuran compound and a low minimum inhibitory concentration (MIC) antibiotic. In some apsects, the disclosed devices can be pre-loaded with an anti-tumor agent and a low minimum inhibitory concentration (MIC) antibiotic.

### Kits

Disclosed herein are kits use in treating a subject suffering from a facultative anaerobe infection. In some aspects, the kits can comprise: (a) a nitroimidazole compound; and (b) a low MIC antibiotic.

Disclosed herein are kits for use in treating a subject suffering from a facultative anaerobe infection. In some aspects, the kits can comprise: (a) a nitrofuran compound; and (b) a low MIC antibiotic.

Disclosed herein are kits for use in treating a subject suffering from a facultative anaerobe infection. In some aspects, the kits can comprise: (a) an anti-tumor agent; and (b) a low MIC antibiotic.

Disclosed herein are kits for use in inhibiting replication of a facultative anaerobe infection. In some aspects, the kits can comprise: (a) a nitroimidazole compound; and (b) a low MIC antibiotic.

Disclosed herein are kits for use in inhibiting replication of a facultative anaerobe infection. In some aspects, the kits can comprise: (a) a nitrofuran compound; and (b) a low MIC antibiotic.

Disclosed herein are kits for use in inhibiting replication of a facultative anaerobe infection. In some aspects, the kits can comprise: (a) an anti-tumor agent; and (b) a low MIC antibiotic.

In some aspects, the nitroimidazole compound can be metronidazole, tinidazole, nimorizole, pimonidazole, or nimorizole. In some aspects, the nitrofuran compound can be furaltadone, nitrofurantoin, nitrofurazone, or furazolidone. In some aspects, the anti-tumor agent can be evofosfamide and tirapazamine, or banoxanthrone. In some aspects, the low MIC antibiotic can be gentamicin, levofloxacin, or vancomycin.

In some aspects, the kits disclosed herein can further comprise one or more of the devices disclosed herein. For example, disclosed herein are kits comprising the Pouch device disclosed herein. In some aspects, the kits disclosed herein can further comprise one or more of the devices disclosed herein preloaded with one or more of the therapeutic agents described herein.

The kits can also comprise suitable instructions (e.g., written and/or provided as audio-, visual-, or audiovisual material). The kits can further comprise one or more of the following: instructions, transfer pipettes, microfuge tubes, plastic sticks (stool pick for solid stool) syringes, a sterile container, delivery devices, tube caps, slides, solid supports, and buffers or other control reagents.

### EXAMPLES

### Example 1: Nitroimidazole Compounds, Nitrofuran Compounds or Anti-Tumor Agents, Combined with Low-MIC Antibiotics to Increase their Efficacy Against Facultative Anaerobe Biofilms

Due to the traumatic nature of battlefield-relevant injuries where bacterial contamination levels can be high, biofilm-related infection is a major concern for military healthcare. Biofilms lead to biofilm-impaired wound healing in soft tissues and difficult-to-treat infections in extremity injuries such as open fractures. A biofilm comprises diverse phenotypic populations, in part, as a response to varied microenvironments throughout the community. As the biofilm grows and matures, nutrients and oxygen become diffusion limited within the core, which by necessity becomes more metabolically quiescent than the biofilm surface where oxygen and nutrients abound. These quiescent biofilm phenotypes can survive antibiotic concentrations much higher than tolerated by actively-replicating (planktonic) bacteria of the same genotype. Antibiotic tolerance of quiescent biofilm bacteria readily exceeds the toxic upper thresholds bounding safe systemic antibiotic doses. Thus, biofilms are a nidus for infection underpinning the disease etiology of the most difficult to treat recalcitrant infections. Biofilms fuel the antibiotic resistance epidemic by permitting the most antibiotic resistant genotypes to survive even aggressive antibiotic treatments and reseed subsequent infection. Described herein are compositions that are more active against S. aureus bacteria in the biofilm core than the metabolically active planktonic phenotype used in the minimum inhibitory concentration (MIC) assay. These compositions outperformed two clinical gold-standard antibiotics (vancomycin and gentamicin) in tests against biofilm bacteria; both of which showed clinically acceptable MICs at 1 μg/ml against the isolate used.

Shortcomings of clinical antibiotic susceptibility assays. The antibiotic-susceptibility assays used to guide clinical treatment and prophylaxis rely on actively-dividing planktonic bacteria (Williams, D. L. & Costerton, J. W. J Biomed Mat Res B 100, 1163-1169 (2011)). Among the assays performed by clinical reference laboratories are the minimum inhibitory concentration (MIC), and disk diffusion (Kirby-Bauer) assays. The ubiquity of these assays is owed to the fact that they are high throughput, relatively quick to perform, repeatable, and have short turnaround times. They inform clinicians regarding which antibiotic are applicable for a given infection from a simple site swab, blood, or aspirate sample. By design, these assays select compounds which stop bacterial replication, they do not necessarily select for compounds which kill quiescent non-replicating phenotypes like those in the biofilm. Thus, when a compound, such as metronidazole performs poorly in an MIC assay, a physician would be thoroughly dissuaded from using it. Likewise, in drug-development, a high MIC value would exclude potentially useful anti-biofilm agents from candidate drug libraries. For comparison, the MIC of metronidazole against S. aureus is >2,500 μg/ml, which is orders of magnitude above a typically acceptable MIC: ˜1 μg/ml or less (FIG. 7). Using orthodox clinical methodology, there would be no basis to administer metronidazole in the treatment of a S. aureus-related infections, despite its exceptional efficacy against bacterial biofilms of the same isolates. The MIC assay fails to provide any information about susceptibility profiles of bacteria that reside in the biofilm phenotype.

Mechanism of hypoxia activated prodrugs in biofilms. The established mechanisms for metronidazole, and like compounds, against known clinical targets (anaerobic bacteria, protozoa, and parasites) give a framework for experimentally unraveling their mechanism in the biofilms of opportunistic pathogens (S. aureus, S. epidermidis, P. aeruginosa, E. coli, K. pneumoniae, A. baumannii, etc.). Metronidazole belongs to the nitroimidazole compound class; these have a strong electron withdrawing nitro functional group central to their mechanism of action. Nitroimidazoles, are prodrugs: they require activation after entering a target cell (FIG. 8). They likely enter a bacterium through passive diffusion but there is some indication that the ferredoxin-linked electron transport systems might play a role in some organisms (nitroimidazoles and bacterial agents). After entering the bacterium, the nitro group is reduced to an anion radical by a nitro reductase enzyme yielding a prodrug radical (Stewart, P. S. & Costerton, J. W. Lancet 358, 135-138, (2001)). Nitroreductases are a ubiquitous family of phylogenetically-related proteins including many oxygen-insensitive NADPH nitroreductases (Bryant, C. & DeLuca, M. J Biol Chem 266, 4119-4125 (1991)). The nitroreductases operating in biofilms of these opportunistic pathogens are unknown, but two identified nitroreductase enzymes in susceptible protozoa and anaerobic bacteria include pyruvate-ferredoxin oxidoreductase and thioredoxin reductase; each have identified homologues in various S. aureus species (UniProt accession #A0A0H2WYT5).

In anoxic conditions the anion nitro-radical forms adjuncts by reacting with nucleophilic groups in proteins and nucleic acids ultimately causing irreparable cellular damage and death (FIG. 8) (Lewis, K. Annu Rev Microbiol 64, 357-372 (2010)). This mechanism of action requires the strong reductive potential of an anaerobic cell. For example, oxygen concentration profiles in P. aeruginosa biofilms tested in air (20.95% oxygen) and 100% oxygen were shown experimentally to approach zero at depths of 50 and 100 μm, respectively (Xu, K. D., et al. Applied and environmental microbiology 64, 4035-4039 (1998)). Oxygen tensions in healthy human tissues are much lower from 1% to 11% and in the surgical site dive still lower as compromised vasculature and infection intensify tissue hypoxia (Epari, D. R., et al. Bone 43, 734-739 (2008); Carreau, A., et al. J Cell Mol Med 15, 1239-1253 (2011); and Gottrup, F. World J Surg 28, 312-315 (2004)). Hypoxia in human wounds inevitably shifts bacterial metabolism towards anaerobic processes, a condition which must be further exacerbated by a bulky biofilm and impeded diffusion created by orthopedic hardware. In the presence of oxygen the prodrug radical is quickly cycled back to the parent compound before it can react directly with cellular species in a process called futile cycling (Lewis, K. Handb Exp Pharmacol, 121-133 (2012)). Although futile cycling in toxic conditions detoxifies the activated prodrug, the cyclical process pumps out levels of superoxide radical anions which can overwhelm some organisms (Docampo, R. & Moreno, S. N. Fed Proc 45, 2471-2476 (1986)). In most organisms, catalase and superoxide dismutase render the radical anion harmless (Jenks, P. J. & Edwards, D. I. Int J Antimicrob Agents 19, 1-7 (2002)).

Regardless of the precise mechanistic details, nitroimidazoles employ radical ions to induce irreparable cellular damage in target organisms. Opportunistic pathogens can draw on a repertoire of cellular machinery and radical scavengers to manage nitrosative and oxidative stressors. These processes are likely used in the biofilm to manage the stresses induced by nitroimidazole compounds. Although the upregulation of appropriate genes and cellular machinery may be retarded in a biofilm with a reduced metabolic state; a condition which is exacerbated when a companion antibiotic blocks gene expression. This might explain, in part, the stronger synergy observed when gentamicin or levofloxacin are paired with hypoxia-activated prodrugs instead of a β-lactam like nafcillin (FIGS. 1 and 7). Gentamicin impairs gene expression by binding to the 30S subunit of the bacterial ribosome and interfering with protein synthesis; levofloxacin prevents RNA production, and therefore protein production, by inhibiting a necessary DNA gyrase; in contrast the β-lactam nafcillin does not prevent gene expression rather it inhibits enzymatic cross-linking of the peptidoglycans required for the new cell walls of replicating bacteria. The diminished capacity for a biofilm to upregulate metabolism might also explain the dose-dependent mechanism of metronidazole. These nitro-containing compounds might, in essence, be forcing the metabolically inactive portions of the biofilm into a heightened metabolic state in order to survive; this forced metabolic activity might be impeded both by nutrient limitations in the biofilm core and some traditional low-MIC antibiotics that prevent gene expression.

For determining the broad-spectrum utility of metronidazole, respective bacterial species were grown into stationary biofilms using a 2- or 3-day species-specific growth protocol in a CDC biofilm reactor on standard 0.5 in polycarbonate coupons. Each reactor holds up to 24 coupons/samples with adherent biofilms. Bacterial bioburden, represented as colony forming units (CFUs), from five reactor samples/coupons (n=5) were quantified by first dispersing the bacterial biofilms in 2 ml of PBS by standardized vortex-sonication technique and serial-dilution plating. Importantly, this quantification technique provides bioburden data with logarithmic sensitivity. The average CFUs of these control biofilms are displayed as black horizontal lines in the subfigures (FIG. 1). The gray shaded regions represent standard deviations.

Three groups of 5 coupons/samples (n=5) were placed into 2 ml treatments either containing 0.5-1 mg/ml of gentamicin, metronidazole, or combinations thereof (in CAMHB) and incubated for 24 h at 37° C. Importantly, the total antibiotic concentration (mg/ml) was fixed for each sample to enable assessment of compound synergy. Gentamicin was substituted for levofloxacin in Staphylococcus epidermidis as this isolate shows complete resistance to gentamicin (FIG. 1).

After the 24 h treatment, the bacteria adhered to coupons were quantified by rinsing the samples very gently in excess PBS followed by vortexing and sonicating to disperse the biofilm in 2 ml PBS and subsequent serial-dilution plating. The bioburden of the adhered bacteria is represented by the blue bars in FIG. 1. The bacterial bioburden remaining in the antibiotic broth, in which the coupons were treated, was also quantified by the vortexing, sonicating, and serial dilution plating technique with additional alternating steps of centrifugally pelleting the bacteria, removing 90% of the solution, replacing 90% of the solution and repeating 2 additional times thereby decreasing the antibiotic in solution below the MIC values (to prevent residual kill on agar plates). Broth bioburden is represented by the red bars in FIG. 1.

For comparison to these biofilm tests with adjunctive metronidazole, the MIC was determined for each organism and each compound tested. The MICs were determined both aerobically (Green) and anaerobically (Blue) as presented in Table 1.

TABLE 1
MICs for the antibiotics used in biofilm tests in FIG. 1 were performed both
aerobically (Blue) and anaerobically (Green). Units are μg/ml of compound.
For compounds listed together, a 1:1 ratio of the compounds was used while
performing the MIC and the reported MIC represents the sum of both compounds.

| Aerobic | | | | | | | |
|---|---|---|---|---|---|---|---|
| MIC | | | | | | | |
| (μg/ml) | | | | | | | |
| Anaerobic | | | | | | | |
| MIC | | MRSA | S. | P. | E. | K. | A. |
| (μg/ml) | S. aureus | USA300 | epidermidis | aeruginosa | coli | pneumoniae | baumannii |
| Metronidazole | 2500 | 1250 | 312.5 | >2500 | 1250 | >2500 | >2500 |
| | >2500 | 1250 | 625 | >2500 | 156.25 | 1250 | N/A |
| Gentamicin | 1 | 2 | — | 2 | 0.5 | 2 | 2 |
| | 8 | 16 | — | 2 | 4 | 4 | N/A |
| 1:1 | 2 | 4 | — | 4 | 1 | 8 | 8 |
| Metro. + | 16 | 32 | — | 2 | 8 | 16 | N/A |
| Gent. | | | | | | | |
| Levofloxacin | — | — | 0.125 | — | — | — | — |
| | — | — | 0.125 | — | — | — | — |
| | — | — | 0.25 | — | — | — | — |
| 1:1 | — | — | 0.25 | — | — | — | — |
| Metro. + | | | | | | | |
| Levo. | | | | | | | |

The results show in the benchtop testing represented in FIG. 1, metronidazole showed a strong synergistic kill against a range of Gram-negative and Gram-positive organisms when paired with the conventional antibiotic gentamicin or levofloxacin (S. epidermidis). This data demonstrates that metronidazole displays cross-species utility against biofilm persisters.

A close comparison of metronidazole combinations in biofilm tests (FIG. 1) against the aerobic and anaerobic MICs suggests that metabolically active log-phase planktonic bacteria are unaffected by metronidazole regardless of anaerobic or aerobic metabolism with the possible exceptions of Klebsiella pneumoniae, and Escherichia coli, which showed a two- and three-fold reduction in MIC, respectively.

To determine if other hypoxia-activated prodrugs, like metronidazole, are active against the problematic biofilm Phenotypes, a series of cross-comparable experiments were developed. Twelve hypoxia activated prodrugs from three categories were tested: nitroimidazoles (same class as metronidazole), nitrofurans, and anti-tumor agents. For the screening, S. aureus ATCC 6538 was used with the best performing compounds subsequently tested against P. aeruginosa ATCC 27853; these pathogens can then be used as inocula for infection development in the animal work.

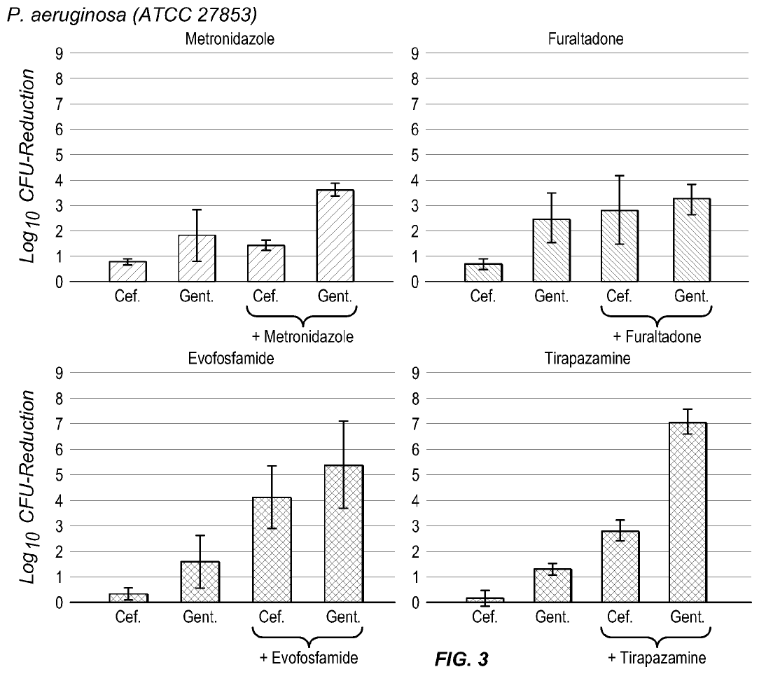

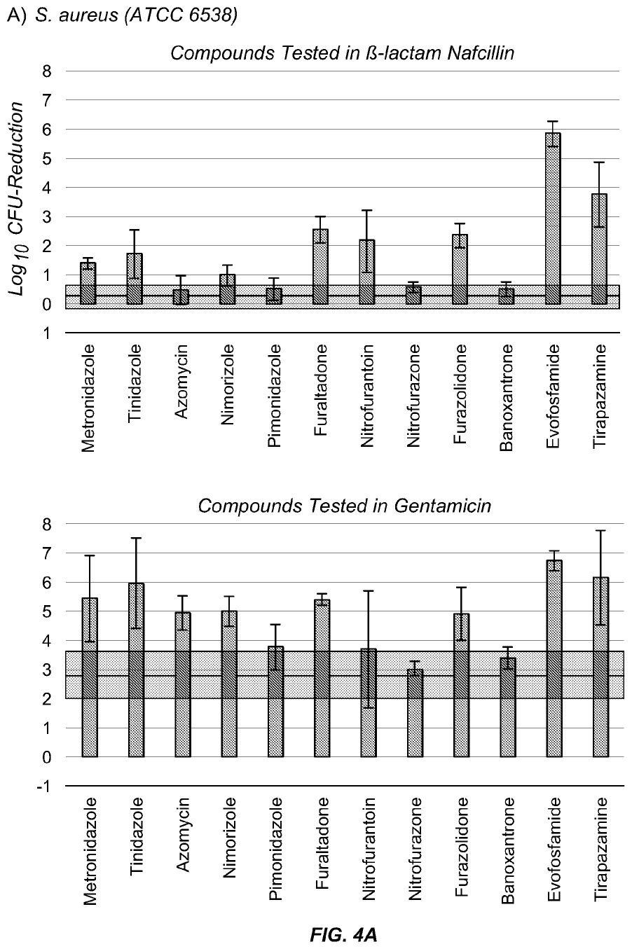

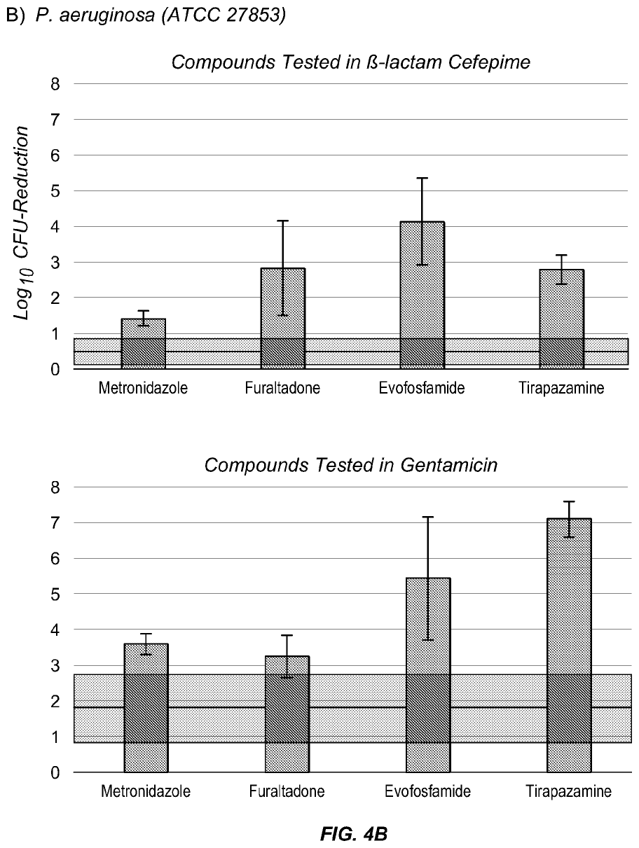

To test each compound, the contents of an entire CDC-biofilm reactor (24 coupons/biofilm samples) were used (see FIG. 2 and FIG. 3). These experiments were designed to have multiple internal controls to enable cross-data comparisons (see FIG. 4). Using the methods described above, 4 reactor samples/coupons served as reactor controls. The remaining 20 samples/coupons were split into 4 treatment groups of 5 samples each (n=5).

For testing against S. aureus biofilms, the compounds were prepared at a concentration of 1 mM in CAMHB; coupons were treated in 2 ml for 24 h. Compounds were intentionally prepared in units of molarity rather than the more common weight/volume ratios of clinicians. This was done to permit comparisons of similar drugs while fixing the total number of relevant functional groups, like the important nitro-group found in most of the hypoxia-activated prodrugs tested.

Each of the 4 treatment groups contained one of the two low-MIC antibiotics: 1 mM nafcillin (a β-lactam antibiotic) or 1 mM gentamicin. This choice was made for two reasons. First, metronidazole has a poor MIC and when tested alone; the biofilm rapidly disperses and aggressively colonizes the treatment broth, quickly depleting nutrients (see the middle column data in FIG. 1). Second, nafcillin did not display synergy nor antagonism with metronidazole whereas gentamicin is synergistic with metronidazole. Against biofilms, β-lactams (like nafcillin) are mostly bacteriostatic in the 24 h test window giving a good backdrop for testing a study compound. Gentamicin in contrast, because of its strong synergy with metronidazole, is an important control for determining if these hypoxia-activated prodrugs need a compound like gentamicin to have the most clinical impact.

Two of the treatment groups were the same for each test: one in 1 mM nafcillin (a β-lactam antibiotic) and a second in 1 mM gentamicin. Likewise, the second two treatment groups had either 1 mM nafcillin or 1 mM gentamicin both with the addition of 1 mM of the compound tested. For comparison for most compounds 1 mM ranges from ˜0.15-0.5 mg/ml, 1 or 2 Log 2 dilutions below tests in FIG. 1. For testing of P. aeruginosa biofilms (FIG. 3), the β-lactam cefepime was substituted for nafcillin due to resistance and the gentamicin concentration was reduced from 1 to 0.1 mM because of excessive kill compared with S. aureus biofilms. The other compounds were tested at the same concentrations.

Quantification of the post-treatment residual bioburden was performed by dispersing biofilm bacteria into the 2 ml treatments with its constituent bacteria by vortexing, and sonicating. The antibiotic was removed from a representative aliquot (1 ml) sample, alternating steps of centrifugally pelleting the bacteria, removing 90% of the solution, replacing 90% of the solution and repeating 2 additional times thereby decreasing the antibiotic in solution below the MIC values. The pelleted bacteria were resuspended and measured by the serial dilution plating technique. Thus, the values presented in FIGS. 2, 3 and 4 represent the cumulative change in bioburden from the surface-attached biofilms and bacteria in the treatments.

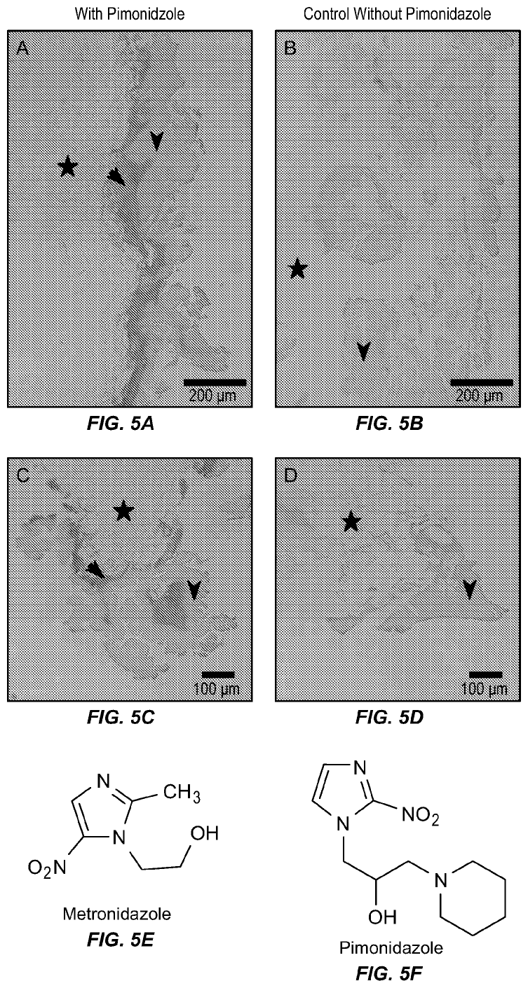

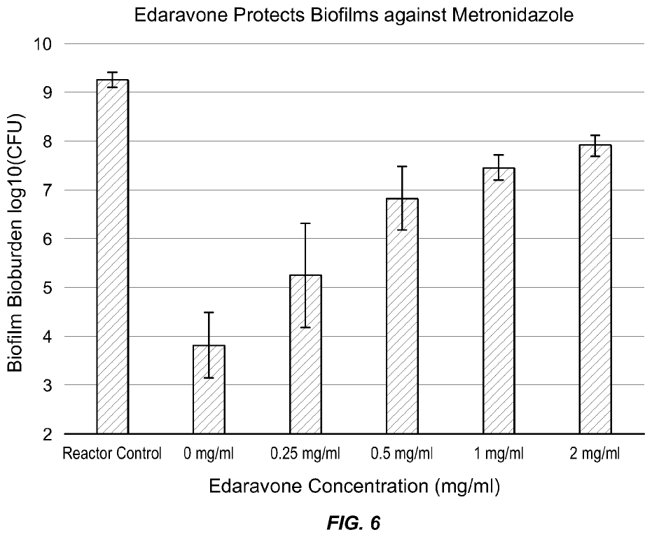

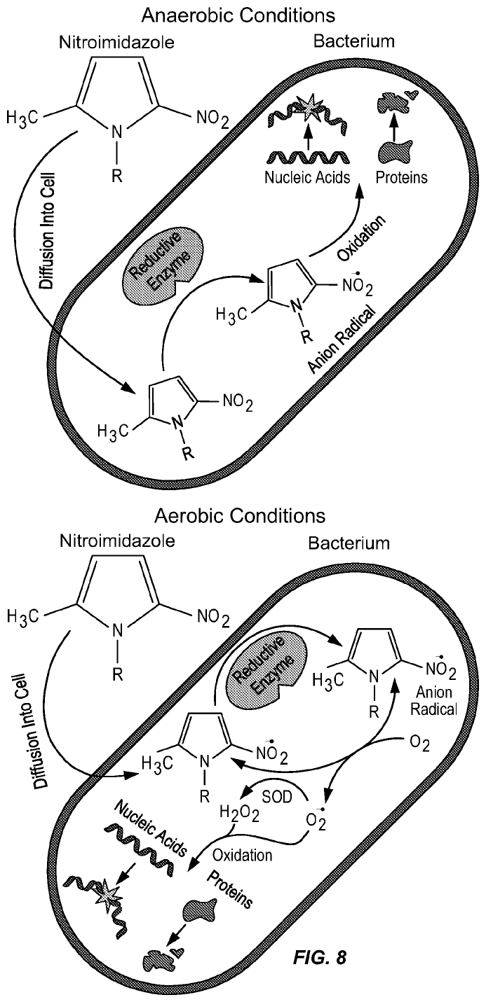

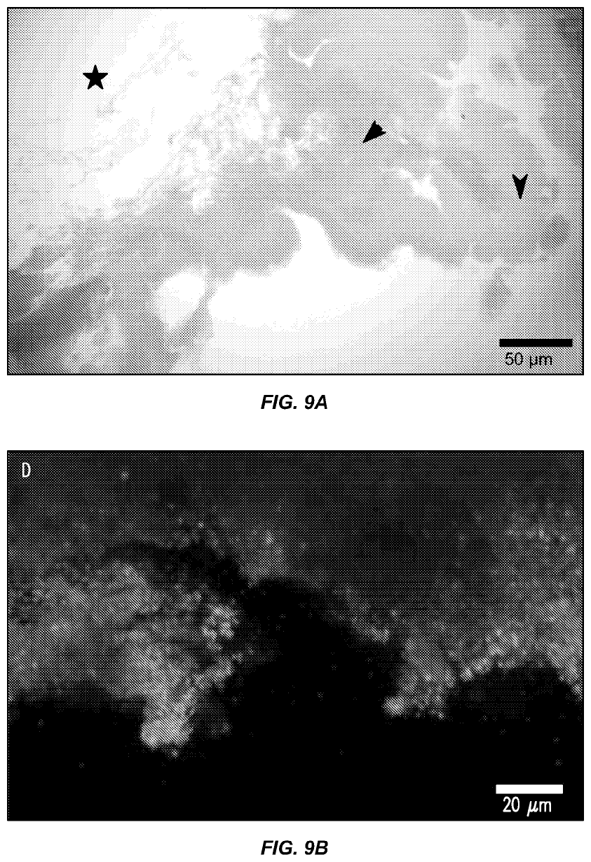

The experimental evidence shows with S. aureus biofilms showing that radical ion species are directly involved in the mechanism of action of nitro-containing compounds; a nitro-reductase enzyme is active in the biofilm; and that the core of the biofilm is under sufficiently high oxygen tension for a nitro-radical to persist (<10 mmHg). In one experiment, S. aureus biofilms were subjected to a bacteriostatic solution of nafcillin (0.25 mg/ml) and an aggressive dose of metronidazole (4 mg/ml). The coupons were divided evenly into 5 groups (n=8) which were treated with serial concentrations of the potent radical scavenger edaravone ranging from 0-2 mg/ml. The radical-scavenger edaravone neutralized the killing-effect of metronidazole in a dose dependent manner over the 24 h experiment (FIG. 6). These data show that the mechanism of metronidazole in biofilms involves a radical ion species that if quenched, neutralizes its killing power. These data demonstrate that an activated prodrug or a superoxide radical anion from futile cycling (see, FIG. 8). In a second experiment, biofilms were treated with a small sublethal dose of the nitroimidazole pimonidazole (0.06 mg/ml). Like the other nitroimidazoles, pimonidazole is reductively activated in hypoxic cells by a nitro-reductase enzyme. In hypoxia, the reduced compound indiscriminately forms adducts with cellular components including adducts with thiol groups which can be recognized with a primary antibody by immunohistochemical staining (FIG. 5A and FIG. 9A) (Masaki, Y. et al. PLOS One 11, e0161639 (2016)). Similarly, in the presence of oxygen (>10 mmHg), the reduced pimonidazole nitro anion is cycled back to the parent compound and pimonidazole adducts do not form. The formation of pimonidazole adducts banded throughout the deepest parts of the biofilm core were observed on sections of pimonidazole treated biofilms (FIG. 5A and FIG. 9A). This immunohistochemical staining shows nitro-reductase activity in the biofilm and provides evidence of sufficiently high oxygen tension in the biofilm for a prodrug radical to persist and react with cellular components. In close agreement, live-dead staining of metronidazole-treated biofilms show an abundance of dead and dying bacteria in the biofilm core contrasted with viable bacteria on the biofilm surface (FIG. 9B). Taken together, these data show that nitro-containing compounds paired with traditional low-MIC antibiotics which stop bacterial gene expression can be useful as antibiofilm agents, and effective in preventing, treating or inhibiting a facultative anaerobe infection, and that their method of action is based on the production of radical species that preferentially kill bacteria in the anoxic metabolically quiescent biofilm core.

The results identified additional compounds from each of the three classes with efficacy against biofilm phenotypes of S. aureus and P. aeruginosa. The majority of the compounds tested showed benchtop anti-biofilm utility against S. aureus compared with antibiotic controls, including nitrofurazone and Banoxantrone.

The nitrofuran furaltadone displayed remarkable efficacy against both S. aureus and P. aeruginosa biofilms compared against the other nitroimidazoles and nitrofurans. This compound also displayed considerable efficacy with β-lactam antibiotics and therefore might be more broadly useful with a wider-range of traditional low-MIC antibiotics.

The anti-tumor agents, Evofosfamide and Tirapazamine, showed significant activity against both S. aureus and P. aeruginosa biofilms.

Next, a time-lapse confocal imaging paired with live-dead staining was used to observe the location of metronidazole-induced cell death.

Further, the Hypoxyprobe™ immunohistochemistry kit was used to identify regions of the biofilm that might be hypoxic, and to identify regions with nitro-reductase activity. At the core of the Hypoxyprobe™ kit is the use of the nitroimidazole pimonidazole; it is used to treat, usually animal tissues, but in this case bacterial tissues (biofilms) while in homeostasis prior to fixation, embedment, sectioning, and staining (see structures FIGS. 5E-F). The nitro group on pimonidazole, like that on metronidazole, is reductively activated in hypoxic mammalian tissues by reaction with catalytic nitro-reductase enzymes; it then forms indiscriminate adducts with inter-cellular thiol groups. In the presence of oxygen, the reduced nitro group in pimonidazole (and metronidazole) rapidly reacts with oxygen thereby cycling back to the parent compound. The Hypoxyprobe™ kit contains a primary antibody against these intercellular pimonidazole adducts. Successful immunohistochemical staining of pimonidazole adducts in the biofilm core indicates two important things. First, it would show nitro-reductase activity in the biofilm, and second it would be direct evidence for sufficiently high oxygen tension for a nitroimidazole prodrug radical to persist and react with critical cellular components.

To perform this staining, S. aureus ATCC 6538 biofilms were grown in a modified CDC-biofilm reactor on coupons aseptically cut from dental collagen plugs (HeliPlug™). The collagen plugs were used because the samples had to be embedded in paraffin and sectioned on a microtome; hard substrates are not amenable to this process. After the 48 h biofilm growth period, n=8 biofilm samples were immersed in 2 ml of 0.06 mg/ml pimonidazole in (CAMHB) for 5 h. An n=8 additional control samples were immersed in 2 ml of CAMHB and incubated for the identical 5 h period. The samples were then fixed in 4% paraformaldehyde, sectioned, stained using a standard colorimetric immunohistochemistry procedure with the provided primary mouse a-pimonidazole antibody, a secondary biotinylated goat a-mouse antibody, and the appropriate horse radish peroxidase and development solutions. Representative micrographs from pimonidazole treated samples and controls are displayed in FIG. 5. This colorimetric dye stains brown. The controls samples, which did not receive pimonidazole treatment, otherwise had the exact same handling and staining steps as the pimonidazole treated samples (FIG. 5).

To aid in the interpretation of these Hypoxyprobe™ experiments, additional data is shown in FIG. 6. In this experiment, S. aureus biofilms grown in a CDC biofilm reactor were subjected to a solution of 0.25 mg/ml nafcillin, serving as a bacteriostatic, and an almost lethal dose of 4 mg/ml metronidazole. The coupons were divided evenly into 5 groups of (n=8). The five groups were dosed with serial concentrations of the potent radical-scavenger edaravone ranging from 0-2 mg/ml (FIG. 6).

The immunohistochemical staining of pimonidazole adducts banding through the deepest parts of the biofilm core indicate that there is indisputable nitro-reductase activity in the biofilm, and provides evidence for sufficiently high oxygen tension for a nitroimidazole prodrug radical to persist and react with important cellular components.

The results also show that the potent radical-scavenger edaravone proportionally negates the killing power of metronidazole. This is further evidence for the classical mechanism of hypoxia-activated prodrugs working in the core of the biofilm as the activated form of these drugs are radical species with reduced nitro groups.

### Example 2: A Device for Delivering High Concentrations of Antibiotics to Musculoskeletal Sites

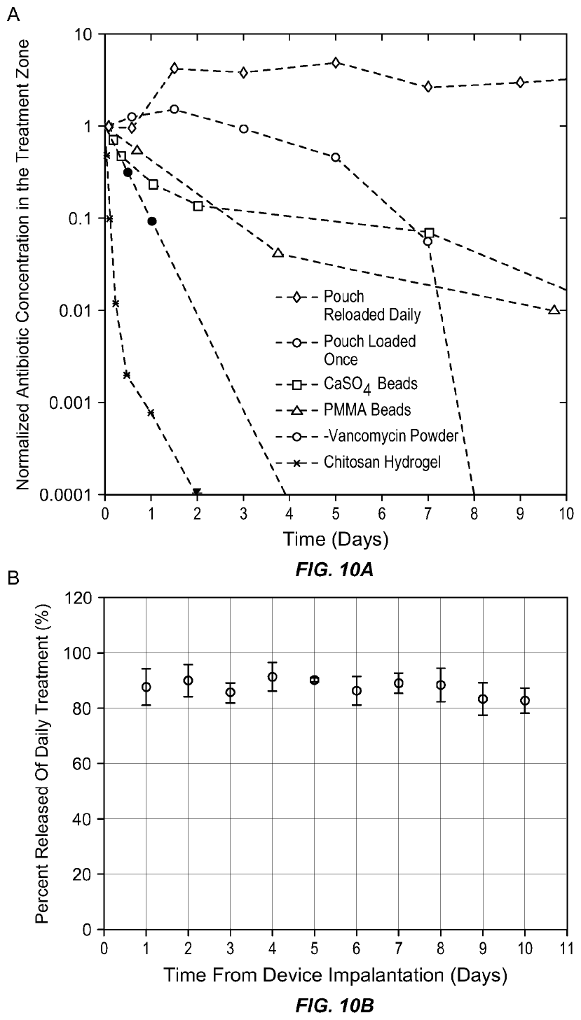

The treatment of musculoskeletal infections usually includes extensive debridement, hardware removal, and long courses of systemic antibiotics. In high-risk procedures, surgeons frequently use one of several off-label strategies in an attempt to achieve high local dose of antibiotics at the site. Several off-label local delivery approaches have gained clinical acceptance yet remain limited in practice. Bone cements are routinely loaded with antibiotics, formed into small beads and packed within a surgical site. Antibiotic beads can be made from both polymethylmethacrylate (PMMA) or resorbable calcium sulfate (CaSO₄) cements. PMMA curing is exothermic, limiting application to heat-stable antibiotics. Tight and rapid polymerization results in antibiotic entrapment; about 3-7% of antibiotic is ever released (Zalavras, C., et al. Clin Orthop Rel Res 427, 86-93 (2004)), peak concentrations are low (5-25 μg/mL) and may be insufficient against biofilms (Moojen, D. J. F. et al. J Arthroplasty 23, 1152-1156 (2008); and Snir, N., et al. Orthopedics 36, e1412-1417 (2013)). CaSO₄ cements accommodate heat-sensitive antibiotics, are biodegradable, and release the antibiotic payload. Yet dissolution of CaSO₄ causes drainage at the implant site, and increases risk of heterotopic ossification (McPherson, E. J., et al. Reconstructive Review, 32-43 (2013); and Helgeson, M., et al. Orthopedics 32, 323 (2009). Additionally surgeons have resorted to peppering wounds and implants with pure antibiotic powder (vancomycin or tobramycin) in hopes of treating and preventing infection (Trujillo, J., et al. Wounds 29, E84-E87 (2017); and Johnson, J. et al. J Arthroplasty 32, 924-928 (2017)). The most important limitation of these off-label approaches is their inability to sustain high antibiotic concentrations at the surgical site. Concentrations in the treatment zone diminish rapidly as lymphatic clearance washes away the antibiotic from every diminishing antibiotic reservoir. The concentration of antibiotic around CaSO₄ beads decrease by ˜90% within 3 days (Aiken, S., et al. Surg Infect (Larchmt) 16, 54-61 (2015)). Antibiotic powders sprinkled in wounds diminish even quicker (FIG. 10); within 1 day, nearly 90% of antibiotic is gone (Working, Z. M., et al. Injury 48, 1459-1465 (2017); LaPlante, K. L. & Mermel, L. A. Antimicrob Agents Chemother 53, 3166-3169 (2009); and Pinheiro, L. et al. Mem Inst Oswaldo Cruz 109, 871-878 (2014)).

To overcome these physical limitations, a versatile device that can sustain local, high dose therapeutics in a surgical site was developed. This device comprises a temporarily implanted (<30 days) antimicrobial-filled tubular reservoir with a permeable rate-determining membrane communicating with a percutaneous refilling port (FIG. 11). Passive diffusion of antibiotics through the rate-determining membrane and daily refilling, maintain high target concentrations of therapeutics locally at the implant site. Once treatment is complete, the device is removed using a minimally invasive procedure by filling with lidocaine to numb the local area, deflating, cutting suture anchors (if used), and extracting through the existing transdermal port incision (<1 cm; FIG. 11). Removal and insertion of the device mirrors procedures familiar to surgeons for placing and removing surgical drains. The benefits of this device (referred to hereafter as the Pouch or ‘the Pouch device”) are four-fold. First, local release of antibiotic(s) to the surgical site maximizes antibiotic coverage where needed while minimizing systemic toxicity. Second, it sustains high dose release via a reloadable/refillable design (FIG. 10). Third, it can be loaded with a variety of antibiotics or combinations thereof to manage a broad scope of indications. Fourth, compound selection can be spontaneously altered, for example if there are adverse reactions do drugs or in the event a lab result suggests a more appropriate antibiotic regimen.

Use of the Pouch device in a sheep model of biofilm-related orthopedic infection. The Pouch device and two clinical standards of care (IV-vancomycin, and gentamicin-loaded Stimulan® beads) were tested in a sheep model of biofilm-related orthoepic infection. Briefly, the proximal medial aspect of the right tibia is subjected to a blast from an air cannon to simulate traumatic soft tissue damage. In a surgical procedure, the periosteum is stripped, and two biofilm-contaminated fixation plates are situated above osteotomized fractures. At necropsy, one of the fixation plates and the surrounding tissue serve for microbiological analysis whereas the plate and tissues of the second site are used for histological analysis. Four groups of sheep (n=5/group) were used in testing. The first group served as a positive control of infection. The Pouch device was tested in one group. The Pouch device was placed during the surgery and refilled daily for 10 days with 480 mg of gentamicin sulfate, 1 g of vancomycin, and 36 mg of rifampin. Ten days after surgery the Pouch device was removed, and the sheep monitored for an additional 11 days; endpoint was 21 days for the groups. For comparison, clinical standards of care were tested in the remaining two groups: IV vancomycin, and Gentamicin-loaded Stimulan® beads. The sheep in the IV vancomycin group received 1 g of vancomycin twice daily (2 g/day) for the first 10 days. In the third group, a 12 cc pack of Stimulan® Rapid Cure beads (CaSO₄) formulated per-manufacturers recommendations with 120 mg of gentamicin sulfate, and packed around the implant site at the time of surgery. The animal groups survived 21 days after the initial procedure.

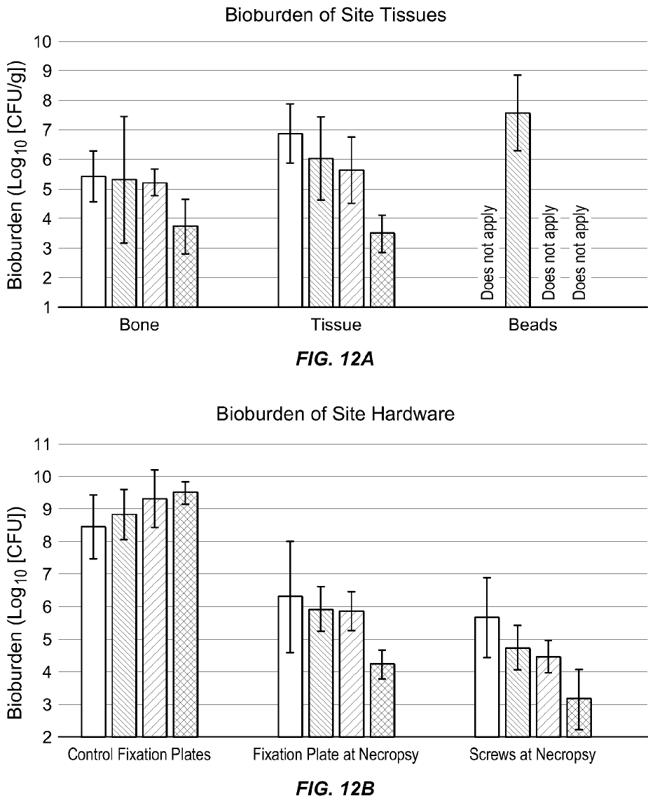

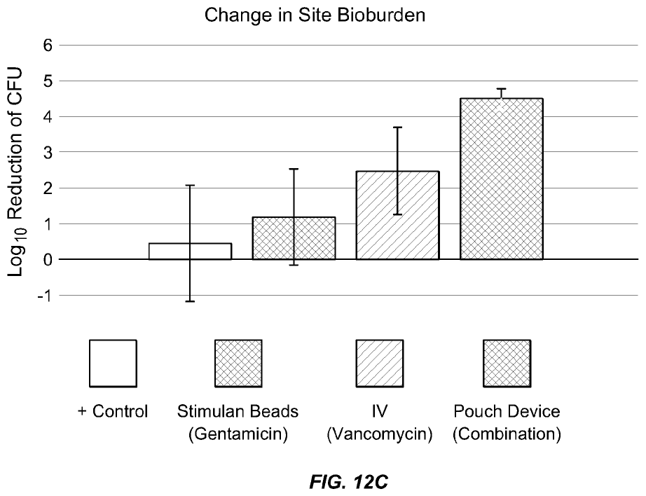

The Pouch device resulted in a 4.5 log₁₀ reduction in bacterial bioburden from the initial inocula, outperforming by a wide margin the clinical standards of care; IV vancomycin and the Stimulan® beads reduced bioburden by 2.5 and 1.2 log₁₀ units, respectively (FIG. 12). The statistical difference between the Pouch device and control group, the Pouch device and IV vancomycin group, and the Pouch device and Stimulan® bead group was p=0.004, p=0.008, and 0.019, respectively. For the Pouch device, the bioburden in the bone and soft tissue surrounding the implant were below the 105 colony forming units (CFU)/g threshold (3.7±0.9 and 3.5=0.6 log₁₀ CFU/g, respectively), which has become a quantitative threshold defining infection (Bowler, P. G. Ostomy Wound Manage 49, 44-53 (2003); and Krizek, T. J. & Robson, M. C. American journal of surgery 130, 579-584, (1975)). The bioburden in the hard and soft tissues of the positive control, IV vancomycin, and Stimulan® groups all exceeded the 105 threshold (FIG. 12). Furthermore, the bioburden levels of the IV vancomycin and Stimulan® were not statistically significantly different compared to the positive control (p=0.13 and 0.69, respectively). Similar results were observed by microbiological analysis of the adherent bacteria on the site hardware: fixation plate and screws. During the necropsy, the bacterial bioburden in residual bio-erodible Stimulan® beads remaining in the implant site was quantified and found to harbor the highest concentration of bacteria of any of the samples analyzed from any of the sheep in this study at 7.6±1.3 log₁₀ (CFU/g). Once the antibiotics leached from these beads, in a process which occurs rapidly after implantation (FIG. 10), bacteria colonized the acellular space aggressively. These antibiotic-loaded beads functioned poorly in this animal model and ultimately served as a nidus of infection.

The Pouch device performed well in this aggressive model of infection despite the exclusive use of traditional low-MIC antibiotics; as discussed, elevated concentrations of low-MIC antibiotics have diminishing returns against the metabolically quiescent biofilm components. The Pouch device is successful because it achieves sustained local, high dose therapy of antimicrobial chemotherapeutics unlike off-label approaches—e.g., antibiotic powders and Stimulan® beads-employed by clinicians in desperation, which too often fail. A release curve was generated from the residual antibiotic solutions which were removed daily while refilling the device (FIG. 10B). The daily percent release ranged within a narrow band from 82.8%-91.4%. Little variation in antibiotic release across the 10-day window in which the devices were implanted was observed. It was found that membrane fouling and fibrotic encapsulation do not affect antibiotic release within the implantation time of this short-duration device.

### Example 3: In Vitro Biofilm Growth and Bioburden Quantification

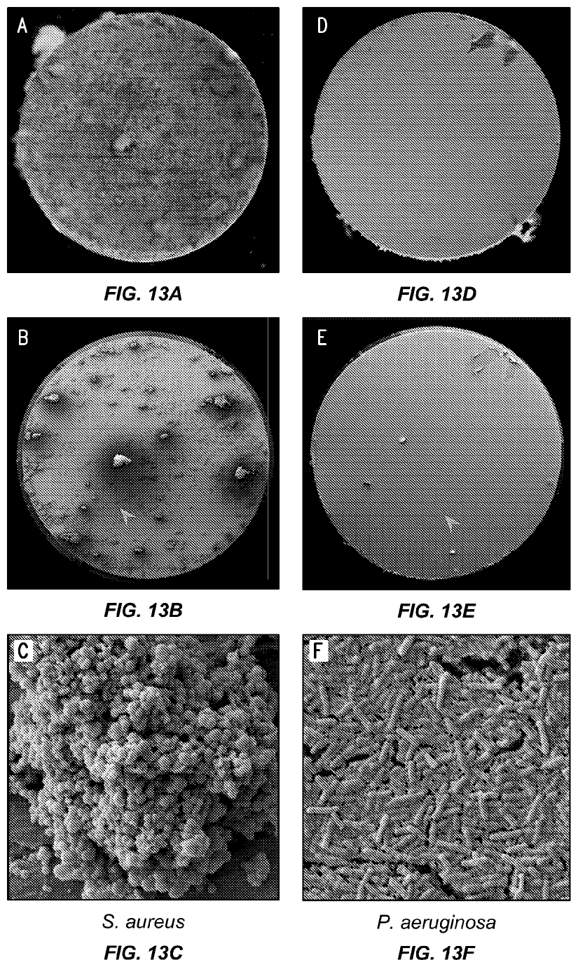

Established protocols (Williams, D. L. & Costerton, J. W. J Biomed Mat Res B 100, 1163-1169 (2011); Williams, D. L. et al. Biomaterials 33, 8641-8656 (2012); Williams, D. L. et al. PLOS One 14, e0206774 (2019); Williams, D. L., et al. Curr Microbiol 62, 1657-1663 (2011); and Rasmussen, R. M., et al. Biofouling (2019)) were used for growing robust biofilms with the most common orthopedic pathogens on a range of substrates (gray traces FIG. 1, see also FIG. 13)—.

Prior to antibiofilm efficacy testing, the MIC of each antibiotic will be determined to confirm bacterial susceptibility to low-MIC antibiotics. Biofilms will then be grown on polycarbonate coupons in a CDC biofilm reactor for susceptibility testing. Polycarbonate coupons are will be used for efficacy screening because they are autoclavable, do not crack glass test tubes due to lower density than metals, easily textured to promote consistent biofilm growth, and are relatively inexpensive. Growth substrate has little, if any, effect on phenotypic tolerance of the adherent biofilm to antibiotics; antibiotic tolerance in biofilms is primarily dependent on surface areal density (biofilm thickness) and biofilm age.

Biofilms will be grown by adding 500 ml of 100% brain heart infusion broth (BHI) to the biofilm reactor, then inoculating the contents with 1 ml of a 0.5 McFarland solution prepared in phosphate buffered saline (PBS) from a fresh culture plate. The reactor and its contents will be incubated on a hotplate at 34° C. for 24 h at 130 RPM stirring speed. After this 24 h batch phase, the reactor will be perfused with 10-20% BHI at 6.4 ml/min for an additional 24 h. This flow phase mimics the replenishing nutrients maturing biofilms receive in wounds by serous discharge and/or breakdown of dead and dying tissues. Each reactor will yield 24 biofilm-covered coupons. Four coupons will be used to obtain baseline growth/coupon and the remainder will be used for testing antibiotic treatments. For testing, each biofilm-covered coupon will be submerged in 2 ml of antibiotic solution prepared in 100% cation-adjusted Mueller Hinton broth (CAMHB). Biofilms will be exposed to treatments for 24 h at 37° C.

Biofilm bioburden will be quantified using an established protocol of vortexing, sonicating, centrifugal pelleting to remove residual antibiotic, followed by log₁₀ serial dilution plating. Briefly, 200 μl sterile glass beads (0.25 mm diameter) will be added to the 2 ml of treatment solution surrounding each coupon. The glass tube containing the treatment, glass beads, and biofilm-covered coupon, will be vortexed for 2 min, sonicated for 10 min, then vortexed again for 10 s. To remove residual antibiotics before serial dilution plating, 1 ml of the dispersed biofilm-treatment solution will be transferred to a sterile microcentrifuge tube and centrifuged at 5000 RPM (˜2000x G) for 3 min; 900 μl of the supernatant will be removed and replaced with sterile PBS and vortexed. This log₁₀ dilutive washing procedure will be repeated until the antibiotic concentration is calculated to be under the MIC value (typically 2-3 washes for most treatments). When the pellet is resuspended for the last time, 100 μl of the sterile glass beads (0.25 mm diameter) will be added to the microcentrifuge tube, followed by vortexing for 2 min, sonicating for 10 min, and then vortexed again for 10 s to completely break up the pellet and suspend the bacteria. The bioburden of viable biofilm constituents will be determined by log₁₀ serial dilution plating.

Low-MIC companion antibiotics for pairing with metronidazole. The ability of hypoxia-activated prodrugs, like metronidazole, to damage and kill biofilm phenotypes is dependent on the antibiotic with which it is paired (FIGS. 1 and 7). Bacterial biofilms will be treated in combinations of metronidazole with a diversity of traditional low-MIC antibiotics selected from across a range of antibiotic classes (Table 2). By choosing representative antibiotics from a distinct antibiotic class, the gamut of antibiotics mechanism will be explored in the context of the antibiofilm agent metronidazole. This data will test whether metronidazole will have strongest synergistic kill against in vitro biofilms when paired with companion antibiotics that mechanistically prevent gene expression.

TABLE 2
Examples of traditional low-MIC antibiotics
that will be selected from major
classes and used in combination with metronidazole.

| | Candidate Compound | Candidate Compound |
|---|---|---|
| Antibiotic Class | (S. aureus) | (P. aeruginosa) |
| β-lactam | Nafcillin | Cefepime |
| Aminoglycoside | Gentamicin | Gentamicin |
| Fluroquinolone | Levofloxacin | Levofloxacin |
| Glycopeptide | Vancomycin | |
| Macrolide | Azithromycin | Azithromycin |
| Lincosamide | Clindamycin | |
| Oxazolidinone | Linezolid | Linezolid |
| Polypeptide | Polymyxin E | Polymyxin E |
| Rifamycin | Rifampin | Rifampin |
| Sulfonamide | Sulfamethoxazole | Sulfamethoxazole |
| Tetracycline | Minocycline | Minocycline |
| Phosphonic | Fosfomycin | Fosfomycin |

Hypoxia-activated prodrugs like metronidazole, have exceptionally high MIC concentrations against the opportunistic pathogens implicated in orthopedic infections (. Because of this, testing of metronidazole against biofilms requires the addition of a low-MIC antibiotic at super MIC concentrations (10-50× the MIC) in every treatment. If a traditional antibiotic is not added, the biofilm bacteria quickly disperse, replicate in log-phase planktonic division, deplete the nutrients in the treatment, and begin to die. Hypoxia-activated prodrugs, for example, β-lactam antibiotics, were shown to be the least synergistic: nafcillin for Gram-positive organisms, and cefepime for Gram-negative organisms. For this reason, β-lactam antibiotics will be used as a treatment control for one group in each reactor to facilitate cross-reactor data comparison. The 24-biofilm-covered coupons from a single reactor will be used in the testing of a single low-MIC candidate antibiotic for use with metronidazole (Table 3). The coupons will be allocated as shown in Table 3. Briefly, four biofilm-covered coupons will serve as a baseline, and the remaining 20 coupons will be divided evenly into 4 treatment groups of n=5/treatment. Two of the groups will be treated with β-lactam one of which will also contain adjunctive metronidazole. The other two groups will be treated with a low-MIC antibiotic from Table 2; one group will also receive adjunctive metronidazole. The low MIC antibiotics, including β-lactams, will be loaded into treatments from 10-50× their MIC. Metronidazole will be loaded into the treatment at a fixed concentration of 1 mM (˜0.17 mg/ml); this concentration is within a range that can be achieved in local tissues using the Pouch device. This testing will use the two representative Gram-negative and -positive organisms: S. aureus and P. aeruginosa.

TABLE 3
Allocation of 24 biofilm-covered coupons in each test.
Allocation of 24 Biofilm-covered Coupons in Each Test

| Group | Replicates (n) |
|---|---|
| Reactor Control | 4 |
| β-lactam Control | 5 |
| (Nafcillin or Cefepime) | |
| Metronidazole + β-lactam | 5 |
| Candidate Antibiotic | 5 |
| Metronidazole + | 5 |
| Candidate Antibiotic | |

Statistical (power) analysis and study design. Sheep will be organized into 3 treatment groups with n=8 sheep/group (Table 4). With a sample size of n=8/group, it is expected that an effect size of 8/8 sheep being infected with greater than 10⁸ CFU in wounds in positive control sheep, and greater than 105 CFU in wounds of sheep treated with standards of care. For sheep treated with the combination of metronidazole and a low-MIC antibiotic, it is expected 0/8 will become infected and have less than 102 CFU in wounds. In the case that 1 without infection and 1 unanticipated with infection, ⅞ (87.5%) vs ⅛ (12.5%), the sample size of n=8 sheep/group provides 87% power using a two-sided Fisher's exact test (Table 5). Basing sample size on a simple comparison of proportions, like the Fisher's exact test, provides a conservative sample size estimate for the Logrank test, so the statistical power will be greater. This is because the Logrank test uses information in the timing of the infections, where the infections in positive controls will likely occur early in follow-up, while infections in sheep treated with the Pouch will likely not occur.

TABLE 4
Groups of sheep, isolate that will be inoculated, and treatments.

| Group | | | | Duration of |
|---|---|---|---|---|
| (n = 8 | | | Antibiotics | Pouch |
| each) | Isolate | Treatment | Used | Placement |
| 1 | S. aureus | Pouch | Low-MIC | 10 Days |
| | | | antibiotic | |
| 2 | S. aureus | Pouch | Metronidazole | 10 Days |
| 3 | S. aureus | Pouch | Low-MIC | 10 Days |
| | | | antibiotic + | |
| | | | Metronidazole | |

TABLE 5
Possible outcomes of infection and the related
power associated with those outcomes.

| Sample Size | Effect Size (fraction of | Power of |
|---|---|---|
| n per group | infection events) | comparison |
| 8 | 0/8 (0%) vs 8/8 (100%) | >0.99 |
| 8 | 1/8 (12.5%) vs 8/8 (100%) | 0.99 |
| 8 | 2/8 (25.0%) vs 8/8 (100%) | 0.88 |
| 8 | 0/8 (0%) vs 7/8 (87.5%) | 0.99 |
| 8 | 1/8 (12.5%) vs 7/8 (87.5%) | 0.87 |
| 7 | 1/7 (14.3%) vs 6/7 (85.7%) | 0.68 |

The data shown in FIG. 12 showed greater than a 4.5 log₁₀ reduction in CFUs using the Pouch device for a 10-day period in vivo. Based on these outcomes and the assumptions used in the power analysis above, it is expected that n=8 sheep/group will provide greater than 80% power to detect a difference amongst comparative groups. Post-monitoring, CFU counts will serve as the primary outcome measure and will be analyzed statistically as outlined and using the parametric t test.

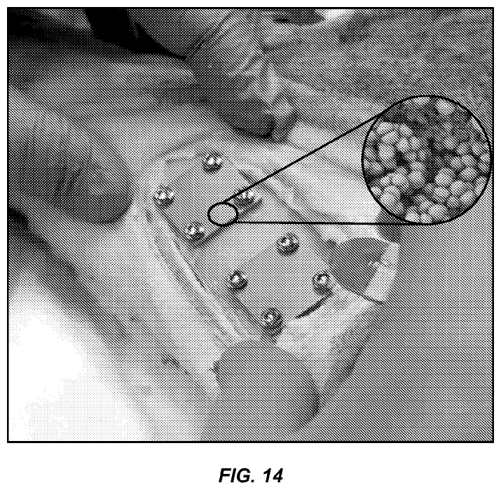

Procedures. Soft tissue trauma will be created to model a component of open fracture/musculoskeletal injury by exposing the proximal medial aspect of a tibia to a high pressure blast of air from an air cannon. To further model an open fracture, the bone will be surgically exposed, periosteum disrupted, and an osteotomized fracture created to a depth of ˜2 mm in an axial direction along host bone that extends roughly 1 mm beyond the borders of implanted titanium plates. Biofilms will be grown on custom titanium fixation plates using modified reactor arms in a CDC biofilm reactor (Williams, D. L., et al. Curr Microbiol 62, 1657-1663 (2011)). The biofilm growth protocols are the same as those described herein for growing biofilms with the CDC biofilm reactor. The biofouled fixation plates will be secured over the osteotomized fractures. In addition to biofilm inocula, sheep will also be inoculated with planktonic bacteria to assess the Pouch device against biofilms and planktonic bacteria that may form a biofilm. After plates are secured, a planktonic culture of bacteria will be adjusted to a 0.5 McFarland standard and a 100 μL aliquot (˜5×10⁶ CFU) will be placed on top of each plate. A Pouch device will be placed in the surgical site in proximity to the stainless-steel plates and the site sutured closed (see, FIG. 14).

After the surgical site is closed, the Pouch device will be filled with 18 mL of antibiotic combination, estimated to include antibiotics in amounts consistent with a recommended daily dose. For example, the pouch might include 480 mg of gentamicin sulfate and/or 500 mg of metronidazole for a sheep weighing 70 kg. Antibiotic solution will be exchanged daily for 10 days. Using an empty Lure Lock syringe, it will be connected to the needleless connector to withdraw old solution. Eighteen ml of fresh solution will be pushed into the Pouch device to fill it again.

The sheep will be monitored for a total of 21 days then euthanized. A clinical grading scale (Williams, D. L. et al. Biomaterials 33, 8641-8656 (2012); Williams, D. L. et al. J Biomed Mat Res A 100, 1888-1900 (2012); and Williams, D. L. et al. Biofilm 2 (2020)) will be used to assess infection development and determine if euthanasia is a required early endpoint. At the time of necropsy, both the study limb will be dissected, and samples of subdermal tissues and host bone will be cultured. Bone will be collected using a trephine and/or ronjeur device, then ground to homogenate and quantified using our serial-dilution protocols above to calculate biofilm burden/g of bone tissue. For each animal, one of the two simulated fracture fixation plates will be processed for quantification to determine CFU/plate, and the other will remain attached to the bone and surrounding soft tissue to be fixed in modified Karnovsky's or 10% formalin and used in subsequent histological analysis.

Anesthesia: Prior to surgery, sheep will be fasted for ˜12 hours. Sheep will be anesthetized using an intravenous (IV) injection of propofol (3-7 mg/kg) to allow for endotracheal tube intubation. Once the induction anesthetic takes effect, an ophthalmic ointment will be administered to each eye to maintain corneal moistness, and the trachea will be intubated for inhalant anesthetic delivery. Anesthesia will be maintained with isoflurane gas (0.5-5.0% to effect) in oxygen, delivered with a re-breathing anesthesia circuit.

Antibiotics: Antibiotics are not intended to be used other than in the Pouch device as it is an infection study to assess the ability of the synergistic antibiotic combination delivered via the Pouch device to eradicate biofilm in the musculoskeletal site and on simulated fracture fixation hardware.

Air Impact Device (AID) Exposure. Each sheep leg will be exposed to a high pressure blast of air using a Martin Engineering Tornado Air Cannon (Williams, D. L. et al. JMIR Res Protoc 8, e12107, doi: 10.2196/preprints. 12107 (2018)). The purpose of traumatizing the leg region is to create soft tissue trauma, which is a component of open fracture injuries.

The proximal medial aspect of the leg will be situated underneath the opening of the AID at a distance of ˜2 inches. The sheep will be protective cloth covering and plugs will be put in its ears. The AID will be pressurized to 100 psi and the pressure released (˜400 lbs of force) (Williams, D. L. et al. JMIR Res Protoc 8, e12107, (2018). This process creates immediate soft tissue trauma as indicated by rupture of blood vessels/capillaries.

Surgical Site Preparation. After AID exposure, animals will be placed under general anesthesia and positioned in sternal recumbency on an operating table. An orogastric “stomach” tube may be inserted to prevent regurgitation aspiration and bloat during the anesthesia period. The right hind limb of each sheep will be circumferentially clipped free of hair/wool, from the hoof to the hip region. Wool will be removed by vacuum and the entire clipped area will be prepped for surgery. The hoof will be isolated in a sterile rubber glove and wrapped with sterile VetWrap. Surgical scrubbing will begin at the center of the proximal medial aspect of the tibia using three (3), two-step cycles consisting of center-out scrubbing with a povidone iodine or chlorhexidine scrub solution and center-out removal with 70% isopropyl alcohol. After a brief dry time, the scrubbed area will be sprayed with povidone iodine solution and allowed to dry. The surgery site will be sterile draped for aseptic surgery and prepped with a final aseptic scrub (e.g. ChloraPrep™).

Biofilm Preparation. Biofilms will be grown on the surface of simulated fracture fixation plates using standard protocols in the Bone and Biofilm Research Lab to more closely model the environment of biofilm-dwelling organisms and a contaminated/infected open fracture site (Williams, D. L. & Costerton, J. W. J Biomed Mat Res B 100, 1163-1169 (2011); and Costerton, J. W. & Irvin, R. T. The Bacterial Glycocalyx in Nature and Disease. Ann Rev Microbiol 35, 299-324 (1981)). Biofilms will be grown on the surface of simulated fracture fixation plates. Growing biofilms on the plates can simulate a fracture site that is in the stage of biofilm-related infection.

Surgical Procedure and Pouch Placement. An incision will be created in the anterior region of the proximal medial aspect of the right tibia. The bone will be surgically exposed, periosteum disrupted using a periosteal elevator and other tools as needed, and an osteotomized fracture created to a depth of ˜2 mm at a 20° angle to the axial direction along host bone that extends roughly 1 mm beyond the borders of implanted metal plates. Osteotomized fractures are always open and can differ in healing compared to closed fractures (Decker, S., et al. Orthop Rev (Pavia) 6, 5575 (2014)). Yet they are common in sheep studies as they provide consistency across groups and have similar healing properties (Decker, S., et al. Orthop Rev (Pavia) 6, 5575 (2014)). A Pouch device will be placed over the simulated fracture fixation plates such that the controlled release membrane is adjacent to the plates. The percutaneous tube will be tunneled and exit through the groin region.

Surgical Site Evaluation and Pain Management: Throughout the course of the study, each sheep will be monitored to assess symptoms of pain and distress. Under veterinary supervision, animals that show signs of pain or distress will be treated with carprofen (4 mg/kg), Buprenex (0.005-0.01 mg/kg), additional fentanyl patches, or another regimen indicated by the veterinarian. NSAIDs will primarily be used to manage pain and swelling. Using a clinical grading system sheep will be monitored daily. Animals will be further monitored for limping, lethargy, irritability, and going “off feed” and/or water. Based on these criteria, a four-tiered clinical grading system will be established (Williams, D. L. et al. Biomaterials 33, 8641-8656 (2012); Williams, D. L. et al. J Biomed Mat Res A 100, 1888-1900 (2012); and Williams, D. L. et al. Acta Biomater, doi: 10.1016/j.actbio.2019.01.055 (2019)). Animals will be monitored for a period of 21 days to allow bone response and determine efficacy/effect of the various parameters.

Bone Labeling. Fluorochrome labeling is a method of measuring the mineral apposition rate (MAR), at which osteoid matrix, produced by osteoblast cells, is deposited and mineralized to form new bone. For this study, calcein fluorochrome will be used as a non-antibacterial agent to label bone and to calculate the MAR, i.e., remodeling rate of the sheep bone, and assess bone viability. As calcein is injected, it is taken up by osteoblast cells and released into the collagen matrix of newly forming bone. After processing, calcein fluorochrome can be observed in tissue samples as they are imaged using an excitation wavelength of 495 nm and emission of 515 nm.

Calcein will be prepared in reverse osmosis water to a final concentration of 30 mg/ml. Approximately ⅓ of the final volume will be sodium hydroxide due to the acidic nature of calcein. The pH will be adjusted to 7.2-7.4, filtered using a 0.22 μm filter for sterility and the solution administered IV at 0.33 ml/kg of body weight. Two separate injections will be given: one 16 days and one 5 days prior to the established end point of each sheep to create a double label in the bone. Sheep euthanized prematurely will not receive calcein injections.

Sample Size Estimate and Statistical Handling. There will be n=8 animals/group. With a sample size of n=8/group, we assume an effect size of 8/8 sheep being infected with greater than 10⁸ CFU in wounds in positive control sheep, and greater than 105 CFU in wounds of sheep that will be treated with standards of care. For sheep treated with the Pouch device, it is expected that 0/8 will become infected and have less than 102 CFU in wounds. To allow for an unanticipated outlier—e.g., 1 without infection and 1 unanticipated with infection, ⅞ (87.5%) vs ⅛ (12.5%), the sample size of n=8 sheep/group provides 87% power using a two-sided Fisher's exact test (Table 4 of Narrative). Basing sample size on a simple comparison of proportions, like the Fisher's exact test, provides a conservative sample size estimate for the Logrank test, so the statistical power will actually be greater. This is because the Logrank test uses information in the timing of the infections, where the infections in positive controls will likely occur early in follow-up, while infections in sheep treated with the Pouch will likely not occur. Post-monitoring, CFU counts will serve as the primary outcome measure and will be analyzed statistically using the parametric t test.

Post-Euthanasia Processing. Microbiological Analysis and Tissue Processing. At necropsy, the limb will be disarticulated, transferred to a biosafety cabinet, the skin prepped for sterile access to the subdermal tissues, a swab of the soft tissue taken, and samples of soft tissue collected and analyzed to determine CFU/g tissue using established techniques. One of the simulated fracture fixation plates and accompanying screws will be removed and likewise quantified to determine CFU/sample. Lastly, bone cores will be collected sterilely and quantified to determine CFU/g bone.

Tissue Embedment/Sectioning. One of the simulated fracture fixation plates will remain in place and will be processed for histological analysis. Samples will be prepared and analyzed by perforating hard and soft tissue to allow for perfusion of fixative. The sample will be fixed in Formalin using 3×24-h changes, and 70% ethanol for 3×24 hours then dehydrated in a Tissue-Tek VIP instrument using ascending concentrations of ethanol (from 70% to 80% to 95% to 100%) and xylene. Bone will then be moved into a solution of 80% methyl methacrylate and 20% n-butyl phthalate (the combination of these two solutions is hereafter referred to as Solution A), and mixed for 5 days to infuse the tissues. After 5 days, Solution A will be poured out and a fresh aliquot of Solution A, mixed with 2.5 g/L of Perkadox 16 (the catalyst for polymerization), will be added to the sample. The sample will be kept in a desiccator at 4° C. for 7 days. Finally, 5 g/L of Perkadox 16 will be added to another batch of fresh Solution A and exchanged for the used mixture in the container and the sample kept in a desiccator at 4° C. for an additional 9 days. Samples will then be placed in a new container and Solution A with 5 g/L of Perkadox 16 added and polymerized in 2 cm layers using ultraviolet light. Polymethyl methacrylate (PMMA) sample will contain the excised tissue.

Once embedded, tissue samples will be cut using a band saw to remove excess PMMA and isolate the area of interest. Samples will be further sectioned into ˜2 mm sections using a diamond blade water saw. Radiographs of the sections will be obtained following the same procedure outlined above. Five sections will be obtained per plate. Two sections will be ground and polished to an optical finish, gold coated and analyzed using SEM backscatter electron (BSE) imaging. The remaining three slides will be mounted to plastic slides and ground to approximately 50-70 μm and analyzed with light microscopy for MAR and histopathological analysis.

SEM Analysis. SEM analysis will be performed to examine bone morphology and ingrowth in the region of interest. BSE images will be collected to examine the varying levels of mineralization and response.

MAR Analysis. MAR data collection will be based on Bloebaum et al. (Bloebaum, R. D., et al. Anat Rec A Discov Mol Cell Evol Biol 281 (2004); and Bloebaum, R. D., et al. J Biomed Mat Res A 81A, 505-514 (2007)). In short, after sample sections ground to ˜50-70 um thickness, images will first be collected using a mercury lamp Nikon Labophot microscope to detect the presence of calcein double-labeled osteons of the host bone. Three slides from each sheep will be analyzed and three osteons per slide will be randomly selected in the cortical/periosteal bone region. A total of seven measurements will be made for each double label using ImagePro Plus software. An area of host bone that is not near the plate will also be examined and the MAR calculated.

Histology: Sample slides will be ground to a thickness of ˜50 μm and stained with Sanderson's Rapid Bone Stain. Slides will be placed in the heated stain solution warmed to 50-55° C. for 90 seconds, rinsed in distilled water and dried. To counter stain, each slide will be placed in acid fuchsin (room temperature) for 15 seconds and dried. Macroscopic images of slides will be collected using a Nikon DSLR Macro Camera (Nikon, Melville, NY, USA). Higher magnification will be collected using a Nikon Eclipse E600 microscope (Nikon Instruments Inc.). Using a modified histopathologic grading scale (Smeltzer, M. S. et al. J Orthop Res 15, 414-421 (1997); Williams, D. L. et al. Biomaterials 33, 8641-8656 (2012); and Williams, D. L. et al. J Biomed Mat Res A 100, 1888-1900 (2012)), slides will be examined to determine level of bone and inflammatory response.

What is claimed is:

1. A method of treating, preventing or inhibiting a facultative anaerobe infection in a subject, the method comprising administering to the subject one or more effective doses of a composition comprising a synergistic combination of a nitroimidazole compound and a low minimum inhibitory concentration (MIC) antibiotic.

2. A method of treating, preventing or inhibiting a facultative anaerobe infection in a subject, the method comprising administering to the subject one or more effective doses of a composition comprising a synergistic combination of a nitrofuran compound and a low minimum inhibitory concentration (MIC) antibiotic.

3. A method of treating, preventing or inhibiting a facultative anaerobe infection in a subject, the method comprising administering to the subject one or more effective doses of a composition comprising a synergistic combination of an anti-tumor agent and a low minimum inhibitory concentration (MIC) antibiotic.

4. The method of claim 1, wherein the nitroimidazole compound is metronidazole, azomycin, secnidazole, ornidazole, dimetriazole, panidazole, fexinidazole, benznidaole, misonidazole, magazol, tinidazole, nimorizole, pretomanid, delamanid, or pimonidazole.

5. The method of claim 2, wherein the nitrofuran compound is difurazone, furazolidone, nifurfoline, nifuroxazide, nifurquinazol, nifurtoinol, nifurzide, nitrofural, furaltadone, ranbezolid, furazidine, furaginum, furylfuramide, nifuratel, nifurtimox, nitrofurantoin, nitrofurazone, or furazolidone.

6. The method of claim 3, wherein the anti-tumor agent is evofosfamide, tirapazamine, or banoxanthrone.

7. The method of any of claim 1, 2, or 3, wherein the low MIC antibiotic is gentamicin, nafcillin, levofloxacin, or vancomycin.

8. The method of any of claim 1, 2, or 3, wherein the facultative anaerobe is a Gram-negative bacterium Gram-negative.

9. The method of claim 8, wherein the Gram-negative bacterium is E. coli, K. pneumoniae, A. baumannii, Proteus, S. typhimurium, B. subtilis; S. pyogenes; S. pneumoniae; E. faecalis; methicillin-resistant S. aureus; S. epidermidis or P. aeruginosa.

10. The method of any of claim 1, 2, or 3, wherein the composition is administered by intravenous injection, intraperitoneal injection or implantation, intramuscular injection or implantation, intrathecal injection, subcutaneous injection or implantation, intradermal injection, lavage, bladder wash-out, suppositories, pessaries, oral ingestion, topical application, enteric application, inhalation, aerosolization or nasal spray or drops, ocular administration, or administration to the ear.

11. The method of any of the preceding claims, wherein the subject is infected or has previously been infected with a Gram-negative bacterium or a Gram-positive bacterium.

12. The method of claim 1, wherein the therapeutically effective dose of the nitroimidazole compound; and MIC antibiotic is in a 1:1 ratio.

13. The method of claim 2, wherein the therapeutically effective dose of the nitrofuran compound and MIC antibiotic is in a 1:1 ratio.

14. The method of claim 3, wherein the therapeutically effective dose of the anti-tumor agent and MIC antibiotic is in a 1:1 ratio.

15. A method of inhibiting, treating or preventing a facultative anaerobe infection in a subject, the method comprising administering to the subject having said infection a plurality of therapeutically effective doses of a composition comprising a synergistic combination of a nitroimidazole compound and a low minimum inhibitory concentration (MIC) antibiotic.

16. The method of claim 15, wherein the plurality of therapeutically effective doses of the composition comprising a synergistic combination of the nitroimidazole compound and the low MIC antibiotic is one or more doses administered per day for two or more days per week.

17. A method of inhibiting, treating or preventing a facultative anaerobe infection in a subject, the method comprising administering to the subject having said infection a plurality of therapeutically effective doses of a composition comprising a synergistic combination of a nitrofuran compound and a low minimum inhibitory concentration (MIC) antibiotic.

18. The method of claim 17, wherein the plurality of therapeutically effective doses of the composition comprising a synergistic combination of the nitrofuran compound and the low MIC antibiotic is one or more doses administered per day for two or more days per week.

19. A method of inhibiting, treating or preventing a facultative anaerobe infection in a subject, the method comprising administering to the subject having said infection a plurality of therapeutically effective doses of a composition comprising a synergistic combination of an anti-tumor agent and a low minimum inhibitory concentration (MIC) antibiotic.

20. The method of claim 19, wherein the plurality of therapeutically effective doses of the composition comprising a synergistic combination of the anti-tumor agent and the low MIC antibiotic is one or more doses administered per day for two or more days per week.

21. The method of any of claims 15-20, wherein dosing is continued for one or more weeks per month.

22. The method of any of claims 15-20, wherein dosing is continued for one or more months per year.

23. The method of any of claims 15-22, wherein the composition is administered by intravenous injection, intraperitoneal injection or implantation, intramuscular injection or implantation, intrathecal injection, subcutaneous injection or implantation, intradermal injection, lavage, bladder wash-out, suppositories, pessaries, oral ingestion, topical application, enteric application, inhalation, aerosolization or nasal spray or drops, ocular administration, or administration to the ear.

24. The method of claim 15, wherein the composition comprising a synergistic combination of the nitroimidazole compound and the low MIC antibiotic is administered to the subject immediately after infection or any time within one day to 5 days after infection or at the earliest time after diagnosis of infection with a facultative anaerobe.

25. The method of claim 17, wherein the composition comprising a synergistic combination of the nitrofuran compound and the low MIC antibiotic is administered to the subject immediately after infection or any time within one day to 5 days after infection or at the earliest time after diagnosis of infection with a facultative anaerobe.

26. The method of claim 19, wherein the composition comprising a synergistic combination of the anti-tumor agent and the low MIC antibiotic is administered to the subject immediately after infection or any time within one day to 5 days after infection or at the earliest time after diagnosis of infection with a facultative anaerobe.

27. The method of any of the preceding claims, wherein the composition is a pharmaceutical composition.

28. The method of claim 27, wherein the pharmaceutical composition further comprises one or more pharmaceutically acceptable excipients, diluents, adjuvants, stabilizers, emulsifiers, preservatives, colorants, buffers, flavor-imparting agents or pharmaceutically acceptable carriers suitable for oral, enteral, mucosal, sub-mucosal, parenteral, intravenous, nasal, ocular, ear or transdermal administration.

29. The method of any of the preceding claims, wherein the composition is manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

30. The method of any of the preceding claims, wherein the composition is formulated as an implantable hydrogel or a hydrogel for transdermal application.

31. A combination therapy comprising a nitroimidazole compound and a low minimum inhibitory concentration (MIC) antibiotic.

32. The combination therapy of claim 31, wherein the nitroimidazole compound and the low MIC antibiotic are provided in a single formulation.

33. The combination therapy of claim 31, wherein the nitroimidazole compound and the low MIC antibiotic are provided separately.

34. A combination therapy comprising a nitrofuran compound and a low minimum inhibitory concentration (MIC) antibiotic.

35. The combination therapy of claim 34, wherein the nitrofuran compound and the low MIC antibiotic are provided in a single formulation.

36. The combination therapy of claim 34, wherein the nitrofuran compound and the low MIC antibiotic are provided separately.

37. A combination therapy comprising an anti-tumor agent and a low minimum inhibitory concentration (MIC) antibiotic.

38. The combination therapy of claim 37, wherein the anti-tumor agent and the low MIC antibiotic are provided in a single formulation.

39. The combination therapy of claim 37, wherein the anti-tumor agent and the low MIC antibiotic are provided separately.

40. A kit for use in treating a subject suffering from a facultative anaerobe infection, the kit comprising: (a) a nitroimidazole compound; and (b) a low minimum inhibitory concentration (MIC) antibiotic.

41. A kit for use in treating a subject suffering from a facultative anaerobe infection, the kit comprising: (a) a nitrofuran compound; and (b) a low minimum inhibitory concentration (MIC) antibiotic.

42. A kit for use in treating a subject suffering from a facultative anaerobe infection, the kit comprising: (a) an anti-tumor agent; and (b) a low minimum inhibitory concentration (MIC) antibiotic.

43. A kit for use in inhibiting replication of a facultative anaerobe infection, the kit comprising: (a) a nitroimidazole compound; and (b) a low minimum inhibitory concentration (MIC) antibiotic.

44. A kit for use in inhibiting replication of a facultative anaerobe infection, the kit comprising: (a) a nitrofuran compound; and (b) a low minimum inhibitory concentration (MIC) antibiotic.

45. A kit for use in inhibiting replication of a facultative anaerobe infection, the kit comprising: (a) an anti-tumor agent; and (b) a low minimum inhibitory concentration (MIC) antibiotic.

46. The kit of claim 40 or 43, wherein the nitroimidazole compound is metronidazole, azomycin, secnidazole, ornidazole, dimetriazole, panidazole, fexinidazole, benznidaole, misonidazole, magazol, tinidazole, nimorizole, pretomanid, delamanid, or pimonidazole.

47. The kit of claim 41 or 44, wherein the nitrofuran compound is difurazone, furazolidone, nifurfoline, nifuroxazide, nifurquinazol, nifurtoinol, nifurzide, nitrofural, furaltadone, ranbezolid, furazidine, furaginum, furylfuramide, nifuratel, nifurtimox, nitrofurantoin, nitrofurazone, or furazolidone.

48. The kit of claim 42 or 45, wherein the anti-tumor agent is evofosfamide and tirapazamine, or banoxanthrone.

49. The kit of any of claims 40-48, wherein the low MIC antibiotic is gentamicin, levofloxacin, or vancomycin.

50. A composition comprising: (a) a nitroimidazole compound, a nitrofuran compound, or an anti-tumor agent; and (b) a low minimum inhibitory concentration (MIC) antibiotic.

51. The composition of claim 50, wherein the composition is formulated as or formulated for: a capsule, a tablet, a gel, a geltab, a liquid, a solid, an elixir, a spray, a powder, a suppository or an implant, a sachet, a lozenge, a freeze-dried composition, or a hydrogel.

52. The composition of claim 51, wherein the composition is impregnated into a plastic material.

53. A method of treating a surface or product to kill or inhibit a facultative anaerobe, the method comprising applying the composition of claim 50 or 51 to a surface or product preserves the surface or product by preventing or inhibiting facultative anaerobe formation or growth by one or more facultative anaerobes.

54. The method of claim 53, wherein the surface being selected from the group consisting of: microcapsules, catheters, wound dressings, implants, wound closures, staples, meshes, controlled drug delivery systems, wound coverings, fillers, sutures, tissue adhesives, tissue sealants, absorbable and non-absorbable hemostats, catheters, wound drainage tubes, arterial grafts, soft tissue patches, gloves, shunts, stents, guide wires and prosthetic devices, contact lens and contact lens storage containers, medical devices, oral swabs, sponges, skin swabs, dental appliances and dental devices, microbial-resistant fabrics and apparel, anti-microbial condoms, surgical gowns, microbial-resistant hospital equipment, anti-microbial paper products, animal care products, antimicrobial plastics, antimicrobial plastic devices, rubbers, appliances with antimicrobial constituents or coatings, food processing equipment, food conveyor belts, food packaging equipment, pet or animal food, pet chew toys, pet or animal water bowls, and floating toys.

55. The method of claim 53, wherein the product being selected from the group consisting of: lipsticks, cosmetics and personal care items, fabric detergents, dish detergents, cleansers, soaps, bubble baths, disinfectants, deodorizers, human and animal foods, beverages, antimicrobial packaging, pharmaceutical products feminine hygiene compositions, vaginal douches, antimicrobial soaps, hand sanitizers, deodorants, antiperspirants dental compositions, toothpastes, mouth rinses and washes medications, athlete's foot treatments, cold sore treatments, herpes virus treatments, medicated chewing gums, wound care compositions, dermatological compositions, acne treatments, skin conditioners, skin moisturizers, anti-wrinkle formulations, skin whiteners, sunscreens, tanning lotions, hair products, shampoos, shower gels, bubble baths, conditioners, shaving creams, spermicides.

56. A method of reducing cumulative bioburden of a facultative anaerobe in a subject, the method comprising administering to the subject one or more effective doses of a composition comprising a synergistic combination of a nitroimidazole compound and a low minimum inhibitory concentration (MIC) antibiotic.

57. A method of reducing cumulative bioburden of a facultative anaerobe in a subject, the method comprising administering to the subject one or more effective doses of a composition comprising a synergistic combination of a nitrofuran compound and a low minimum inhibitory concentration (MIC) antibiotic.

58. A method of reducing cumulative bioburden of a facultative anaerobe in a subject, the method comprising administering to the subject one or more effective doses of a composition comprising a synergistic combination of an anti-tumor agent; and a low minimum inhibitory concentration (MIC) antibiotic.

59. The method of claim 56, wherein the nitroimidazole compound is metronidazole, tinidazole, nimorizole, pimonidazole, or nimorizole.

60. The method of claim 57, wherein the nitrofuran compound is furaltadone, nitrofurantoin, nitrofurazone, or furazolidone.

61. The method of claim 58, wherein the anti-tumor agent is evofosfamide and tirapazamine, or banoxanthrone.

62. The method of any of claims 56-58, wherein the low MIC antibiotic is gentamicin, nafcillin, levofloxacin, or vancomycin.
